SEQUENCE LISTING (6 sequences)

SEQ ID NO: 1 - protein, 14 aa, Homo sapiens
CAGGSGGGADGLTF

SEQ ID NO: 2 - protein, 11 aa, Homo sapiens
CAVSGYSTLTF

SEQ ID NO: 3 - protein, 13 aa, Homo sapiens
CAVPYNQGGKLIF

SEQ ID NO: 4 - protein, 13 aa, Homo sapiens
CSARDGTGNGYTF

SEQ ID NO: 5 - protein, 13 aa, Homo sapiens
CASSVGGTDTQYF

SEQ ID NO: 6 - protein, 13 aa, Homo sapiens
CASSLTRTDTQYF

(19) United States
(12) Patent Application Publication
(10) Pub. No.: US 2026/0036592 A1
(43) Pub. Date: Feb. 5, 2026

(54) DETECTION OF ANTI-VIRAL CDR3s IN CANCER

(71) Applicant: UNIVERSITY OF SOUTH FLORIDA, TAMPA, FL (US)

(72) Inventors: George Blanck, Tampa, FL (US); Nandini Goel, Tampa, FL (US); Taha Ibrahim Huda, Tampa, FL (US); Rahul Jain, Tampa, FL (US); Veda Naga Priya Vangala, Tampa, FL (US); Madeline Claire Baker, Tampa, FL (US); Joanna Jiaqi Song, Tampa, FL (US); Boris Chobrutskiy, Portland, OR (US); Andrea Chobrutskiy, Portland, OR (US); Michael J. Diaz, Tampa, FL (US); Utsav Kapoor, Tampa, FL (US); Srijit Paul, Tampa, FL (US)

(21) Appl. No.: 19/285,798

(22) Filed: Jul. 30, 2025

Related U.S. Application Data
(60) Provisional application No. 63/677,021, filed on Jul. 30, 2024.

(51) Int. Cl. G01N 33/68; G01N 33/569; G01N 33/574

(52) U.S. Cl. CPC G01N 33/6857; G01N 33/56994; G01N 33/57407; G01N 2333/05

(57) ABSTRACT

The present disclosure relates to methods, systems and compositions for determining and improving overall survival probabilities in EBV-positive cancer subjects and the role of tumor infiltrating lymphocytes in cancer immunotherapy.

### CROSS REFERENCE TO RELATED APPLICATIONS

This application claims the benefit of priority U.S. Provisional Patent Application No. 63/677,021, filed on Jul. 30, 2024, the disclosure of which is expressly incorporated by reference herein in its entirety.

### REFERENCE TO SEQUENCE LISTING

The sequence listing submitted on Jul. 30, 2025, as an .XML file entitled “11001-228US1_ST26” created on Jul. 30, 2025, and having a file size of 6,038 bytes is hereby incorporated by reference pursuant to 37 C.F.R. § 1.52(e)(5).

### FIELD

The present disclosure relates to methods, systems, and compositions for determining and improving overall survival probabilities in EBV-positive cancer subjects, and the role of tumor-infiltrating lymphocytes in cancer immunotherapy.

### BACKGROUND

Epstein-Barr virus (EBV), a ubiquitous gamma herpesvirus, is implicated in a broad spectrum of human cancers, including Burkitt lymphoma (BL), Hodgkin lymphoma, nasopharyngeal carcinoma, and certain gastric carcinomas. In EBV-positive tumors, the virus persists within malignant cells and expresses latent viral proteins that may serve as antigens capable of eliciting host immune responses. Yet, despite the immunogenic nature of these viral elements, patient outcomes vary widely suggesting differences in the quality, specificity, or functionality of the tumor-infiltrating immune repertoire.

In particular, EBV-positive Burkitt lymphoma is a highly aggressive B-cell malignancy with distinct geographical and molecular subtypes. While some patients respond well to standard chemotherapy, others experience rapid disease progression or relapse, highlighting a need for better prognostic tools and more personalized therapeutic strategies. Although prior studies have described broad associations between immune cell infiltration and clinical outcomes, such findings often lack specificity.

High-throughput sequencing technologies have opened new avenues for profiling T-cell receptor (TCR)/B-cell receptor (BCR) repertoires directly from tumor specimens. Tools that extract TCR/BCR sequences from RNA or DNA data, coupled with reference databases cataloging antigen-specific TCRs/BCRs, have enabled partial reconstruction of the tumor-immune landscape. However, most existing approaches rely heavily on exact sequence identity matching between patient-derived TCRs/BCRs and previously annotated clones. This limits their utility to known TCR/BCR-epitope pairs and fails to account for biochemically plausible but non-identical interactions.

Moreover, few methods link these molecular interactions to meaningful clinical outcomes such as overall survival (OS). There remains a significant gap in the art for approaches that can evaluate immune receptor function based on structural or chemical complementarity to viral epitopes in virally driven cancers like EBV-positive BL and correlate such profiles with patient prognosis or treatment response.

Existing methods do not comprehensively capture receptor-epitope interactions that are functionally relevant but sequence-divergent. Tools for linking immune cell specificity to survival outcomes remain underdeveloped, particularly in the context of EBV-associated malignancies. There is a lack of scalable frameworks that integrate immune receptor profiling with viral epitope databases and statistical models of clinical outcome.

With the advent of large collections of adaptive immune receptor recombination reads representing cancer, there is the opportunity to further investigate the adaptive immune response to viruses in the cancer setting. This is a particularly important goal due to longstanding but still not well-resolved questions about viral etiologies in cancer and viral infections as comorbidities.

Overall, there is a significant need for a novel strategy for assessing viral infection complications in EBV-positive cancer patients and thereby providing effective adaptive immune responses. Therefore, what is needed are new methods for diagnosing and treating cancer patients. The methods disclosed herein address these and other needs.

### SUMMARY

Disclosed herein are methods for determining overall survival and guiding treatment decisions in subjects with Epstein-Barr virus (EBV)-positive cancers by analyzing tumor-infiltrating lymphocyte (TIL)-derived complementarity determining region 3 (CDR3) sequences for reactivity against EBV, including immunotherapies based on sequence matching and chemical complementarity between host immune receptors and EBV epitopes.

In one example, disclosed herein is a method of determining overall survival in a subject with Epstein-Barr virus (EBV)-positive cancer, comprising:
a) obtaining a biological sample from the subject, wherein the biological sample comprises tumor-infiltrating lymphocytes (TIL);
b) extracting complementarity determining region 3 (CDR3) amino acid (AA) sequences from the TIL, thereby obtaining extracted TIL CDR3 AA sequences; and
c) identifying an exact match of extracted TIL CDR3 AA sequences to known anti-EBV CDR3 AA sequences from the biological sample; thereby obtaining an exact match anti-EBV TIL CDR3 AA sequence,

wherein the presence of the exact match anti-EBV TIL CDR3 AA sequence in the biological sample is correlated to higher overall survival compared to a reference control, wherein the reference control does not have the exact match anti-EBV TIL CDR3 AA sequence in the biological sample.

In some examples, the biological sample comprises blood or tumor biopsy.

In some examples, the TILs comprise B cells or T cells.

In some examples, the T cell comprises T cell receptor (TCR), wherein the TCR comprises an alpha chain or a beta chain.

In some examples, the B cell comprises B cell receptor (BCR), wherein the BCR comprises two heavy chains (IGH) and two light chains (IGL).

In some examples, the EBV-positive cancer comprises nasopharyngeal carcinoma (NPC), Hodgkin lymphoma, Burkitt lymphoma, post-transplant lymphoproliferative disorder (PTLD), gastric carcinoma, or ovarian cancer.

In one example, disclosed herein is a method of determining overall survival in a subject with an EBV-positive cancer, comprising:
a) obtaining a biological sample from the subject, wherein the biological sample comprises tumor-infiltrating lymphocytes (TIL);
b) extracting complementarity determining region 3 (CDR3) amino acid (AA) sequences from the TIL, thereby obtaining extracted TIL CDR3 AA sequences; and
c) obtaining a chemical complementarity score (CS) by interacting known EBV epitopes AA sequences to the extracted TIL CDR3 AA sequences,

wherein a high CS is correlated to better overall survival compared to a reference control, wherein the reference control has low CS.

In some examples, the CS is calculated using hydrophobic interactions, electrostatic interactions, or a combination thereof.

In some examples, the biological sample comprises blood or tumor biopsy.

In some examples, the TILs comprise B cells or T cells.

In some examples, the T cell comprises T cell receptor (TCR), wherein the TCR comprises an alpha chain or a beta chain.

In some examples, the B cell comprises B cell receptor (BCR), wherein the BCR comprises two heavy chains (IGH) and two light chains (IGL).

In some examples, the EBV-positive cancer comprises nasopharyngeal carcinoma (NPC), Hodgkin lymphoma, Burkitt lymphoma, post-transplant lymphoproliferative disorder (PTLD), gastric carcinoma, or ovarian cancer.

In one example, disclosed herein is a method of treating an EBV-positive cancer in a subject, comprising:
a) obtaining a biological sample from the subject, wherein the biological sample comprises tumor-infiltrating lymphocytes (TIL);
b) extracting complementarity determining region 3 (CDR3) amino acid (AA) sequences from the TIL, thereby obtaining extracted TIL CDR3 AA sequences;
c) identifying an exact match of extracted TIL CDR3 AA sequences to known anti-EBV CDR3 AA sequences from the biological sample; thereby obtaining an exact match anti-EBV TIL CDR3 AA sequence; and
d) administering a pharmaceutically effective amount of an anti-EBV therapeutic to the subject with a presence of the exact match anti-EBV TIL CDR3 AA sequence,

wherein the presence of the exact match anti-EBV TIL CDR3 AA sequence in the sample is correlated to better overall survival.

In some examples, the biological sample comprises blood or tumor biopsy.

In some examples, the TILs comprise B cells or T cells.

In some examples, the T cell comprises T cell receptor (TCR), wherein the TCR comprises an alpha chain or a beta chain.

In some examples, the B cell comprises B cell receptor (BCR), wherein the BCR comprises two heavy chains (IGH) and two light chains (IGL).

In some examples, the anti-EBV therapeutic comprises an anti-viral therapeutic against EBV epitopes, an adoptive T cell therapy targeting EBV epitopes, a single-chain variable fragment (scFv) targeting EBV epitopes, or an anti-EBV antibody.

In some examples, the anti-EBV therapeutic is administered intravenously, intramuscularly, intraperitoneally, intradermally, or subcutaneously to the subject.

In some examples, the EBV-positive cancer comprises nasopharyngeal carcinoma (NPC), Hodgkin lymphoma, Burkitt lymphoma, post-transplant lymphoproliferative disorder (PTLD), gastric carcinoma, or ovarian cancer.

### BRIEF DESCRIPTION OF THE DRAWINGS

The accompanying figures, which are incorporated in and constitute a part of this specification, illustrate several examples described below.

FIGS. 1A and 1B show overall survival (OS) distinctions between EBV-positive, Ugandan CGCI-BLGSP cases with TCR CDR3 exact amino acid (AA) sequence matches to anti-EBV TCR CDR3s (from the VDJdb) versus cases lacking exact AA sequence matches to anti-EBV CDR3s. FIG. 1A shows a black line, 12 Ugandan CGCI-BLGSP cases with TRA CDR3s that match anti-EBV TRA CDR3s from the VDJdb; gray line, 16 Ugandan CGCI-BLGSP cases with no TRA CDR3 anti-EBV TRA CDR3 matches. Log rank p-value=0.027. FIG. 1B shows a black line, 14 Ugandan CGCI-BLGSP cases with TCR (TRA+TRB) CDR3 anti-EBV TCR CDR3 match; gray line, 14 Ugandan CGCI-BLGSP cases with no TCR CDR3 anti-EBV TCR CDR3 match. Log rank p-value=0.13.

FIG. 2 shows box and whisker plots for a comparison of CSs representing EBV-positive, Ugandan CGCI-BLGSP cases with or without exact AA sequence matches to anti-EBV TCR CDR3s. Box and whisker plot of the maximum Hydro, Electrostatic, and Combo CSs for each Ugandan CGCI-BLGSP case with TCR CDR3s anti-EBV TCR CDR3 match compared to no anti-EBV TCR CDR3 match cases. The notation “+” signifies an anti-EBV TCR CDR3 exact AA sequence match and “−” means no anti-EBV TCR CDR3 match in the following figure legend text. A shows TRA+Hydro (mean of case-maximum values=8.08); B shows TRA−Hydro (mean=7.65); C shows TRA+Electrostatic (mean=3.67); D shows TRA−Electrostatic (mean=3.33); E shows TRA+Combo (mean=9.71); F shows TRA−Combo (mean=9.12); G shows TRB+Hydro (mean=8.21); H shows TRB−Hydro (mean=7.34); I shows TRB+Electrostatic (mean=3.84); J shows TRB−Electrostatic (mean=3.42); K shows TRB+Combo (mean=9.31); L shows TRB−Combo (mean=8.55).

FIGS. 3A and 3B show OS probability distinctions associated with Electrostatic CSs for EBV-positive, Ugandan CGCI-BLGSP cases with TCR CDR3s and IEDB T-cell, EBV epitopes 27992 and 53128. FIG. 3A shows a black line, upper 50th percentile CGCI-BLGSP TCR CDR3-T-cell EBV epitope 27992 Electrostatic CS group; gray line, lower 50th percentile CGCI-BLGSP TCR CDR3-EBV epitope 27992 Electrostatic CS group. Log rank p-value=0.0021. FIG. 3B shows a black line, upper 50th percentile CGCI-BLGSP TCR CDR3-T-cell EBV epitope 53128 Electrostatic CS group; gray line, lower 50th percentile CGCI-BLGSP TCR CDR3-EBV epitope 53128 Electrostatic CS group. Log rank p-value=0.00027.

FIGS. 4A-4D show OS probability distinctions associated with Electrostatic and Combo CSs for EBV-positive, Ugandan CGCI-BLGSP cases with TCR CDR3s and IEDB EBV epitopes representing both T-cell and B-cell assays: IEDB-16878, 429189, 194260, 30951. FIG. 4A shows a Black line, upper 50th percentile CGCI-BLGSP TCR CDR3-EBV epitope 16878 Electrostatic CS group; gray line, lower 50th percentile CGCI-BLGSP TCR CDR3-EBV epitope 16878 Electrostatic CS group. Log rank p-value=0.017. FIG. 4B shows a black line, upper 50th percentile CGCI-BLGSP TCR CDR3-EBV epitope 429189 Electrostatic CS group; gray line, lower 50th percentile CGCI-BLGSP TCR CDR3-EBV epitope 429189 Electrostatic CS group. Log rank p-value=0.015. FIG. 4C shows a black line, upper 50th percentile CGCI-BLGSP TCR CDR3-EBV epitope 194260 Electrostatic CS group; gray line, lower 50th percentile CGCI-BLGSP TCR CDR3-EBV epitope 194260 Electrostatic CS group. Log rank p-value=0.017. FIG. 4D shows a black line, upper 50th percentile CGCI-BLGSP TCR CDR3-EBV epitope 30951 Combo CS group; gray line, lower 50th percentile CGCI-BLGSP TCR CDR3-EBV epitope 30951 Combo CS group. Log rank p-value=0.065.

FIGS. 5A and 5B show OS probability distinctions associated with Electrostatic CSs for EBV-positive, Ugandan CGCI-BLGSP cases with TCR CDR3s and IEDB B-cell, EBV epitopes 95676, 118800. FIG. 5A shows a black line, upper 50th percentile CGCI-BLGSP TCR CDR3-B-cell EBV epitope 95676 Electrostatic CS group; gray line, lower 50th percentile CGCI-BLGSP TCR CDR3-B-cell EBV epitope 95676 Electrostatic CS group. Log rank p-value<0.0001. FIG. 5B shows a black line, upper 50th percentile CGCI-BLGSP TCR CDR3-B-cell EBV epitope 118800 Electrostatic CS group; gray line, lower 50th percentile CGCI-BLGSP TCR CDR3-B-cell EBV epitope 118800 Electrostatic CS group. Log rank p-value=0.0021.

FIGS. 6A-6H show Kaplan-Meier (KM) analyses comparing overall survival (OS) and disease-specific survival (DSS) of case IDs positive versus negative for virus-specific anti-TCR CDR3s recovered from TCGA-OV tumor and blood samples (combined). FIG. 6A shows overall survival (OS) among case IDs positive for anti-EBV CDR3s (arrowhead, black, n=41; median survival, 60.6 months) compared to case IDs negative for anti-EBV CDR3s (grey, n=212; median survival, 39.4 months). Log-rank p value=0.004. FIG. 6B shows disease-specific survival (DSS) among case IDs positive for anti-EBV CDR3s (arrowhead, black, n=40; median survival, 65.5 months) compared to case IDs negative for anti-EBV CDR3s (grey, n=195; median survival, 42 months). Log-rank p value=0.007. FIG. 6C shows overall survival (OS) among case IDs positive for anti-CMV CDR3s (arrowhead, black, n=80; median survival, 49.6 months) compared to case IDs negative for anti-CMV CDR3s (grey, n=173; median survival, 39.6 months). Log-rank p value=0.48. FIG. 6D shows disease-specific survival (DSS) among case IDs positive for anti-CMV CDR3s (arrowhead, black, n=73; median survival, 51.9 months) compared to case IDs negative for anti-CMV CDR3s (grey, n=162; median survival, 42 months). Log-rank p value=0.492. FIG. 6E shows overall survival (OS) among case IDs positive for anti-INFA CDR3s (arrowhead, black, n=21; median survival, 60.6 months) compared to case IDs negative for anti-INFA CDR3s (grey, n=232; median survival, 43.4 months). Log-rank p value=0.226. FIG. 6F shows disease-specific survival (DSS) among case IDs positive for anti-INFA CDR3s (arrowhead, black, n=20; median survival, 60.6 months) compared to case IDs negative for anti-INFA CDR3s (grey, n=215; median survival, 44.3 months). Log-rank p value=0.317. FIG. 6G shows overall survival (OS) among case IDs positive for anti-SARS-CoV-2 CDR3s (arrowhead, black, n=40; median survival, 55.2 months) compared to case IDs negative for anti-SARS-CoV-2 CDR3s (grey, n=213; median survival, 41.4 months). Log-rank p value=0.125. FIG. 6H shows disease-specific survival (DSS) among case IDs positive for anti-SARS-CoV-2 CDR3s (arrowhead, black, n=38; median survival, 55.2 months) compared to case IDs negative for anti-SARS-CoV-2 CDR3s (grey, n=197; median survival, 43.9 months). Log-rank p value=0.254.

FIGS. 7A-7D show Kaplan-Meier (KM) analyses of overall survival (OS), disease-specific survival (DSS), disease-free survival (DFS), and progression-free survival (PFS) in TCGA-OV tumor samples, comparing case IDs positive versus negative for anti-EBV TCR CDR3s. FIG. 7A shows overall survival (OS) among case IDs positive for anti-EBV CDR3s (arrowhead, black, n=9; median survival, 107.92 months) compared to case IDs negative for anti-EBV CDR3s (grey, n=135; median survival, 36.2 months). Log-rank p value=0.011. FIG. 7B shows disease-specific survival (DSS) among case IDs positive for anti-EBV CDR3s (arrowhead, black, n=9; median survival, 107.2 months) compared to case IDs negative for anti-EBV CDR3s (grey, n=121; median survival, 41.1 months). Log-rank p value=0.023. FIG. 7C shows disease-free survival (DFS) among case IDs positive for anti-EBV CDR3s (arrowhead, black, n=8; median survival, 55.2 months) compared to case IDs negative for anti-EBV CDR3s (grey, n=54; median survival, 19.6 months). Log-rank p value=0.034. FIG. 7D shows progression-free survival (PFS) among case IDs positive for anti-EBV CDR3s (arrowhead, black, n=9; median survival, 55.2 months) compared to case IDs negative for anti-EBV CDR3s (grey, n=135; median survival, 18 months). Log-rank p value=0.017.

FIG. 8 shows KM PFS analysis of case IDs positive for anti-EBV TCR CDR3s from RNAseq files representing TCGA-OV samples. Case IDs positive for anti-EBV CDR3s (arrowhead, black, n=71; median survival, 26.9 months); case IDs negative for anti-EBV CDR3s (grey, n=123; median survival, 19 months). Log-rank p value=0.0476.

FIGS. 9A-9D show Kaplan-Meier (KM) analyses of case IDs stratified by upper and lower 50^{th} percentile groups for TRB CDR3-ICP27 Epitope*193996 Combo complementarity scores (CSs). FIG. 9A shows overall survival (OS) among case IDs in the upper 50^{th} percentile of CSs (arrowhead, black, n=196; median survival, 49.3 months) compared to those in the lower 50^{th} percentile (grey, n=198; median survival, 41.6 months). Log-rank p value=0.0084; univariate Cox regression p-value=0.0001. FIG. 9B shows disease-free survival (DFS) among case IDs in the upper 50^{th} percentile of CSs (arrowhead, black, n=94; median survival, 23.1 months) compared to those in the lower 50^{th} percentile (grey, n=97; median survival, 18.0 months). Log-rank p value=0.007. FIG. 9C shows disease-specific survival (DSS) among case IDs in the upper 50^{th} percentile of CSs (arrowhead, black, n=182; median survival, 57.1 months) compared to those in the lower 50^{th} percentile (grey, n=183; median survival, 43.7 months). Log-rank p value=0.008. FIG. 9D shows progression-free survival (PFS) among case IDs in the upper 50^{th} percentile of CSs (arrowhead, black, n=196; median survival, 19.2 months) compared to those in the lower 50^{th} percentile (grey, n=198; median survival, 16.4 months). Log-rank p value=0.03.

FIGS. 10A-10D show KM analyses of case IDs representing the upper or lower 50th percentile groups for TCGA-OV IGH CDR3-IEDB*30951 Hydro CSs. Note, the IGH recombination reads representing the indicated CDR3s for this analysis were obtained from the RNAseq files. FIG. 10A shows Overall Survival (OS): Case IDs representing the upper 50th percentile CSs (Methods) (black, n=91; median survival, 55.2 months); case IDs representing the lower 50th percentile CSs (grey, n=91; median survival, 39.4 months). p-value=0.028; FIG. 10B shows disease-free survival (DFS): Case IDs representing the upper 50th percentile CSs (black, n=46; median survival, 26.7 months); case IDs representing the lower 50th percentile CSs (grey, n=46; median survival, 19.2 months). p-value=0.078. FIG. 10C shows progression-free survival (PFS): Case IDs representing the upper 50th percentile CSs (black, n=91; median survival, 22.3 months); case IDs representing the lower 50th percentile CSs (grey, n=91; median survival, 16.6 months). p-value=0.034. FIG. 10D shows disease-specific survival (DSS): Case IDs representing the upper 50th percentile CSs (black, n=85; median survival, 57.4 months); case IDs representing the lower 50th percentile CSs (grey, n=85; median survival, 43.4 months). p-value=0.049.

FIGS. 11A and 11B show KM analyses of case IDs representing the upper or lower 50th percentile groups for TCGA-OV IGH CDR3-IEDB*30951 Hydro CSs. Note, this analysis represents the IGH CDR3s from an independent research group, in which the recombination reads were mined from TCGA-OV RNAseq files per the distinct Thorsson algorithm. FIG. 11A shows OS: Case IDs representing the upper 50th percentile CSs (Methods) (black, n=94; median survival, 55.2 months); case IDs representing the lower 50th percentile CSs (grey, n=95; median survival, 43.2 months). p-value=0.052; FIG. 11B shows DSS: Case IDs representing the upper 50th percentile CSs (black n=86; median survival, 58.1 months); case IDs representing the lower 50th percentile CSs (grey, n=86; median survival, 44.5 months). p-value=0.043.

FIG. 12 shows KM analysis of case IDs representing the upper or lower 50th percentile groups of Hydro CSs for CGCI-BLGSP IGH CDR3-IEDB*86944. OS: Case IDs representing the upper 50th percentile CSs (Methods) (black, n=19); case IDs representing the lower 50th percentile CSs (grey, n=20). p-value=0.068.

FIGS. 13A-13C show KM analyses of case IDs representing the upper or lower 50th percentile groups of Hydro CSs for NCICCR-DLBCL IGH CDR3s and multiple IEDB epitopes. FIG. 13A show IEDB*144799 OS: Case IDs representing the upper 50th percentile CSs (Methods) (black, n=54); case IDs representing the lower 50th percentile CSs (grey, n=55). p-value=0.029. FIG. 13B shows IEDB*87359 OS: Case IDs representing the upper 50th percentile CSs (black, n=54); case IDs representing the lower 50th percentile CSs (grey, n=55). p-value=0.069. FIG. 13C shows IEDB*134679 OS: Case IDs representing the upper 50th percentile CSs (black, n=54); case IDs representing the lower 50th percentile CSs (grey, n=55). p-value=0.031. FIG. 13D shows IEDB*86900 OS: Case IDs representing the upper 50th percentile CSs (black, n=54); case IDs representing the lower 50th percentile CSs (grey, n=55). p-value=0.044.

### DETAILED DESCRIPTION

Epstein-Barr virus (EBV) is a human herpes virus that is saliva-transmissible and universally asymptomatic. It was first isolated from B-cell lymphoma in 1964. The viral activity phase positive rate of EBV-DNA in children is 11.5%. EBV infects more than 95% of adults and establishes a lifelong infection. EBV seroprevalence may be associated with socioeconomic and racial/ethnic differences. EBV has been linked to infectious mononucleosis (IM), nasopharyngeal carcinoma (NPC), Burkitt lymphoma, gastric cancer, multiple sclerosis (MS), and other multiple diseases. Patients undergoing solid-organ transplantation or hematopoietic stem cell transplantation (HSCT) may be at risk for posttransplant lymphoproliferative disorders (PTLDs) and even death due to reactivation of EBV.

In a healthy body, the immune system can efficiently identify and remove most other viruses and harmful tumor cells by recognizing antigenic substances and activating lymphocytes to protect the host. However, in some cases, EBV can escape the body's immune surveillance, causing a variety of serious diseases. The cytotoxic T lymphocytes (CTLs) are one of the major effector cells in the acquired immune system and have high specificity and killing capacity. CTL-killing is a highly sensitive and rapid process that kills target cells directly. EBV-CTL cellular immunotherapy has been applied in the treatment of diseases caused by EBV. Encouraged by the striking results of chimeric antigen receptor (CAR) T cells therapy targeting B-cell antigens, CAR-T cell therapy targeting EBV antigens is also under development.

EBV is a double-stranded DNA virus. It was the first tumor-related DNA virus found in humans. Similar to other herpesviruses, EBV has a characteristic three-layered configuration: an outer lipid bilayer envelope with viral glycoproteins responsible for recognizing and membrane fusion, the inner pseudoicosahedral nucleocapsid with a 172-kb double-strand DNA genome, and an intermediate pleomorphic episomal compartment with 20-40 different viral proteins. EBV has high variability, and different variants have distinct pathogenicity and regional distribution. The genes encoding the latent membrane protein (LMP) LMP1, EBV nuclear antigen (EBNA) EBNA2 and EBNA3 protein families were found to be the largest number of variants in the EBV genome, followed by BDLF3, which encodes glycoprotein gp150, BLLF1, which encodes gp350/220, BNLF2α and BZLF1, and BRRF2.

Adaptive immune receptors (IRs) have a hypervariable complementarity determining region-3 (CDR3) representing the amino acid (AA) sequence spanning the somatically occurring, recombination joining of the IR V- and J-gene segments. This CDR3 is highly important for antigen binding, and because of the known association between EBV and EBV-positive cancers, the relationship between the presence of anti-EBV CDR3s of T-cell receptors (TCRs) in the blood/tumor and overall survival (OS) is investigated in the current invention. Disclosed herein are the examples strongly indicating TCR AA sequences sourced from blood exome files and their usefulness in prognosis, or patient survival rates in tumors related to viral infections. The anti-tumor immune response is considered to be due to the tumor infiltrating lymphocytes that bind to tumor antigens, which can be either wild-type, early stem cell proteins, presumably foreign to a developed immune system; or mutant peptides, foreign to the immune system because of a mutant amino acid or an otherwise somatically altered amino acid sequence. Disclosed herein are novel methods for assessing the complementarity of tumor mutant peptides and complementarity determining regions (CDRs) of T cell receptors, B cell receptors, and antibodies, based on the retrieval of CDR3 amino acid sequences from both tumor specimen and patient blood exomes and by using a process of assessing CDR3s and mutant amino acid electrical charges. It is shown herein that high electrostatic complementarity and hydropathy values are associated with higher survival rates. In addition, the approach shown herein leads to the identification of genes contributing significantly to the complementary, TCR CDR3, mutant amino acids. The data shown herein indicate a novel approach to tumor immunoscoring and uses thereof for diagnosing, monitoring, and treating cancers. These methods are also used for the identification of high priority neo-antigen, peptide vaccines for treating cancers and/or to the identification of ex vivo stimulants of tumor infiltrating lymphocytes.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “about” and “approximately” are defined as being “‘close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (1) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). “Complementarity Determining Regions” or “CDRs” refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. CDR3s are most variable, of which the tertiary structure determines antigen recognition of an antibody. Framework Regions (FRs) refer to amino acid sequences interposed between CDRs.

As used herein, the term “antibody or a functional fragment thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain antigen recognition property are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or functional fragments thereof” are conjugates of antibody fragments and antigen binding proteins (single chain antibodies). The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

The term “cancer” or “neoplasms” used herein meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genito-urinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples and are also disclosed.

“Complementarity Determining Regions” or “CDRs” refer to amino acid sequences which together define the binding affinity and specificity of the natural variable domain of a native binding site of an antibody, a BCR, or a TCR. The extent of CDRs have been precisely defined and identified by methods known in the arts, such as “Sequences of Proteins of Immunological Interest,” E. Kabat et al., U.S. Department of Health and Human Services, (1991); Wu T T, Kabat E A. “An analysis of the sequences of the variable regions of Bence Jones proteins and myeloma light chains and their implications for antibody complementarity”. J Exp Med (1970); “Canonical structures for the hypervariable regions of immunoglobulins”. Chothia C, Lesk A M J Mol Biol. 1987 Aug. 20; 196(4):901-17, which are incorporated herein by reference for all purposes. In some examples, a CDR begins by the second cysteine in the variable domain, and at the end by the first amino acid in the conserved Phe/Trp-Gly-X-Gly J-region motif.

A “composition” is intended to include a combination of active agent and another compound or composition, inert (for example, a detectable agent or label) or active, such as an adjuvant.

As used herein, the terms “determining,” “measuring,” and “assessing,” and “assaying” are used interchangeably and include both quantitative and qualitative determinations.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein the term “encoding” refers to the inherent property of specific sequences of nucleotides in a nucleic acid, to serve as templates for synthesis of other molecules having a defined sequence of nucleotides (i.e. rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as antigen binding and antigen recognition.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length Win the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “specific binding” refers to the ability of an antigen-binding protein (e.g., an antibody) to preferentially bind to a particular analyte that is present in a homogeneous mixture of different analytes. In certain examples, a specific binding interaction will discriminate between desirable and undesirable antigen in a sample, in some examples more than about 10 to 100-fold or more (e.g., more than about 1000- or 10,000-fold). In certain examples, the affinity between an antigen-binding protein (e.g., an antibody, TCR, or BCR) and an antigen when they are specifically bound in an antigen-binding protein/antigen complex is characterized by a KD (dissociation constant) of less than 10-6 M, less than 10-7 M, less than 10-8 M, less than 10-9 M, less than 10-9 M, less than 10-11 M, or less than about 10-12 M.

The term “subject” or “host” refers to any individual who is the target of administration or treatment. The subject can be a vertebrate, for example, a mammal. Thus, the subject can be a human or veterinary patient. The term “patient” refers to a subject under the treatment of a clinician, e.g., physician. The subject can be either male or female.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder (e.g., a cancer), or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition (e.g. cancer). Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, “EBV-positive cancers” refer to cancers where the Epstein-Barr virus (EBV) is present and thought to play a role in their development. EBV is an oncogenic virus, meaning it can potentially cause cancer. EBV-positive cancers include but are not limited to nasopharyngeal cancer, certain lymphomas (including Burkitt lymphoma and Hodgkin lymphoma), and gastric cancer.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Methods and Systems for Immune Profiling Using TCR Recombination Reads

As used herein, the term “T cell receptor (TCR) recombination read” refers to a sequencing read derived from genomic or transcriptomic data that contains a segment of a recombined T-cell receptor gene, including the variable (V), diversity (D), joining (J), and constant (C) regions. This recombination event is characteristic of mature T-cells and can be computationally recovered from whole exome sequencing (WXS) or RNA sequencing (RNAseq) data.

As used herein, the term “TRA and TRB recombination reads” refers to the subset of TCR recombination reads specifically originating from the alpha (TRA) and beta (TRB) chains of the T-cell receptor complex. These are derived from V(D)J recombination in the TCRA and TCRB gene loci, respectively.

As used herein, the term “CDR3” refers to the complementarity-determining region 3, a hypervariable region of the TCR that is primarily responsible for antigen specificity. The CDR3 region lies at the junction of the V, (D), and J gene segments and is the primary site of contact with a peptide-MHC complex. In some examples, the CDR3 is computationally identified from translated TRA or TRB sequences.

As used herein, the term “whole exome sequencing (WXS) file” refers to a digital file containing high-throughput DNA sequence reads of the protein-coding regions (exons) of the genome. These files are often in FASTQ, BAM, or CRAM format and are used in the present disclosure as a source for extracting TCR recombination reads.

As used herein, the term “RNAseq file” refers to a digital file derived from RNA sequencing, which captures the transcriptome of a biological sample. These files can contain spliced reads and are used for identifying expressed TCR recombination events, especially TRA and TRB transcripts.

“Complementarity Determining Regions” or “CDRs” refer to amino acid sequences which together define the binding affinity and specificity of the natural Fv region of a native binding site of an immunoglobulin or a TCR. The light (L) and heavy (H) chains of an immunoglobulin each have three CDRs, designated L-CDR1, L-CDR2, L-CDR3 and H-CDR1, H-CDR2, H-CDR3, respectively. For an αβTCR, the alpha chain and beta chain each have three CDRs. Accordingly, in some examples, the CDR is a CDR1 of a light chain of an antibody. In some examples, the CDR is a CDR2 of a light chain of an antibody. In some examples, the CDR is a CDR3 of a light chain of an antibody. In some examples, the CDR is a CDR1 of a heavy chain of an antibody. In some examples, the CDR is a CDR2 of a heavy chain of an antibody.

In some examples, the CDR is a CDR1 of an alpha chain of a TCR. In some examples, the CDR is a CDR2 of an alpha chain of a TCR. In some examples, the CDR is a CDR3 of an alpha chain of a TCR. In some examples, the CDR is a CDR1 of a beta chain of a TCR. In some examples, the CDR is a CDR2 of a beta chain of a TCR. In some examples, the CDR is a CDR3 of a beta chain of a TCR.

In some examples, disclosed herein is a method for predicting the CDR is a CDR of a γ chain of a γδTCR. In some examples, the CDR is a CDR of a δ chain of a γδTCR.

In some examples, the nucleic acid is any preceding aspect is a DNA or an RNA. In some examples, the nucleic acid is a DNA. In some examples, the nucleic acid is an RNA. In some examples, the polynucleotide is a DNA. In some examples, the polynucleotide is an RNA. In some examples, the DNA comprises an exon and an intron. In some examples, the DNA is an exon.

It should be understood and herein contemplated that a polypeptide's net charge depends on the number of the charged amino acids the polypeptide contains and the pH of the environment. At physiological pH (pH 7.4), for example, five amino acid residues out of the 20 common amino acids can be charged: two are negative charged: aspartic acid (Asp, D) and glutamic acid (Glu, E) (acidic side chains), and three are positive charged: lysine (Lys, K), arginine (Arg, R) and histidine (His, H) (basic side chains). The term “net charge per residue” of a polypeptide (e.g. a CDR domain) in a certain pH environment is calculated as dividing the overall charge of the polypeptide in such pH environment by the number of amino acid residues of the polypeptide.

In some examples, the reference control is the complementarity score of a CDR domain and a protein associated with a cancer for the lowest 10%, 20%, 30%, 40%, or 50% of the complementarity score of a reference population of patient samples having the cancer. In some examples, the reference control is the complementarity score of a CDR domain and a protein associated with a cancer for the highest 10%, 20%, 30%, 40%, or 50% of the complementarity score of a reference population of patient samples having the cancer. Accordingly, the subject has a shorter overall survival if the complementarity score is lower in the biological sample derived from the subject compared to the reference control, and the subject has a longer overall survival if the complementarity score is higher in the biological sample derived from the subject compared to a reference control.

As used herein, the term “exact match” refers to a perfect alignment between a recovered TCR CDR3 amino acid sequence and a known anti-viral TCR sequence stored in a curated database (e.g., VDJdb), with no mismatches, insertions, or deletions at any position within the sequence.

As used herein, the term “anti-viral TCR CDR3” refers to a TCR CDR3 amino acid sequence that has been experimentally verified or computationally inferred to recognize a viral peptide, as cataloged in reference databases such as VDJdb. These may be specific to viruses such as Epstein-Barr Virus (EBV), cytomegalovirus (CMV), Influenza A (INFA), or SARS-CoV-2.

In one example, disclosed herein is a method of determining overall survival in a subject with Epstein-Barr virus (EBV)-positive cancer, comprising:
obtaining a biological sample from the subject, wherein the biological sample comprises tumor-infiltrating lymphocytes (TIL);
extracting complementarity determining region 3 (CDR3) amino acid (AA) sequences from the TIL, thereby obtaining extracted TIL CDR3 AA sequences; and
identifying an exact match of extracted TIL CDR3 AA sequences to known anti-EBV CDR3 AA sequences from the biological sample; thereby obtaining an exact match anti-EBV TIL CDR3 AA sequence,
wherein the presence of the exact match anti-EBV TIL CDR3 AA sequence in the biological sample is correlated to higher overall survival compared to a reference control, wherein the reference control does not have the exact match anti-EBV TIL CDR3 AA sequence in the biological sample.

In some examples, the biological sample comprises blood or tumor biopsy.

In some examples, the TILs comprise B cells or T cells.

In some examples, the T cell comprises T cell receptor (TCR), wherein the TCR comprises an alpha chain or a beta chain.

In some examples, the B cell comprises B cell receptor (BCR), wherein the BCR comprises two heavy chains (IGH) and two light chains (IGL).

In some examples, the EBV-positive cancer comprises nasopharyngeal carcinoma (NPC), Hodgkin lymphoma, Burkitt lymphoma, post-transplant lymphoproliferative disorder (PTLD), gastric carcinoma, or ovarian cancer.

In one example, disclosed herein is a method of determining overall survival in a subject with an EBV-positive cancer, comprising:
obtaining a biological sample from the subject, wherein the biological sample comprises tumor-infiltrating lymphocytes (TIL);
extracting complementarity determining region 3 (CDR3) amino acid (AA) sequences from the TIL, thereby obtaining extracted TIL CDR3 AA sequences; and
obtaining a chemical complementarity score (CS) by interacting known EBV epitopes AA sequences to the extracted TIL CDR3 AA sequences;
wherein a high CS is correlated to better overall survival compared to a reference control, wherein the reference control has low CS.

In some examples, the CS is calculated using hydrophobic interactions, electrostatic interactions, or a combination thereof.

As used herein, the term “chemical complementarity score (CS)” refers to a computational score that estimates the potential molecular compatibility between a TCR CDR3 sequence and a peptide epitope, based on physicochemical properties. The score may include components such as: Hydrophobic CS: reflects hydrophobic interaction potential, Electrostatic CS: reflects charge-based interactions, and Combo CS: a combined metric integrating hydrophobic and electrostatic properties to model overall binding likelihood.

As used herein, the term “viral epitope” refers to a short peptide sequence derived from a viral protein that is presented by major histocompatibility complex (MHC) molecules and recognized by TCRs. Viral epitopes used in the present disclosure are obtained from immune databases such as the Immune Epitope Database (IEDB).

As used herein, the term “adaptive match” refers to a bioinformatic platform or tool (e.g., adaptivematch.com) that allows input of TCR and epitope sequences and outputs chemical complementarity scores, along with survival-based stratification analytics, including Kaplan-Meier (KM) survival plots and Cox regression results.

As used herein, the term “survival analysis” refers to statistical techniques for evaluating the time until an event of interest (e.g., death, relapse) occurs. In some examples of the present disclosure, survival analysis includes the generation of Kaplan-Meier plots, Cox proportional hazards models, and p-value calculations comparing groups stratified by TCR features such as match status or complementarity score.

As used herein, the term “upper and lower 50th percentile” refers to the division of subject cases based on the distribution of a numeric feature (e.g., chemical complementarity score), where the upper 50th percentile represents the top half of scores and the lower 50th percentile represents the bottom half. These groups are compared to assess differential survival outcomes.

As used herein, “overall survival” or “OS” refers to the length of time from diagnosis or the start of treatment until death from any cause. OS is a comprehensive clinical outcome that does not distinguish cause of death.

As used herein, “disease-specific survival” or “DSS” refers to the length of time from diagnosis or treatment initiation until death caused specifically by the diagnosed disease, such as ovarian cancer. Patients who die from other causes are censored in this analysis.

As used herein, “disease-free survival” or “DFS” refers to the time after primary treatment during which a patient survives without any signs or symptoms of the disease. It is used to measure the efficacy of treatment in preventing recurrence.

As used herein, “progression-free survival” or “PFS” refers to the time during which the patient survives with the disease but without clinical or radiographic evidence of disease progression. It is frequently used in oncology trials where complete eradication of disease may not be possible.

As used herein, the term “adaptive match” refers to a bioinformatics tool or platform capable of accepting TCR CDR3 sequences and peptide epitope sequences as input, computing chemical complementarity scores, and optionally returning survival-based stratification data including Kaplan-Meier p-values, hazard ratios, and percentile-based groupings.

In one example, disclosed herein is a method of predicting overall survival (OS) in a subject having Epstein-Barr virus (EBV)-positive Burkitt lymphoma (BL), comprising: obtaining a biological sample comprising tumor-infiltrating T cells from the subject; determining the presence of at least one T cell receptor (TCR) complementarity determining region 3 (CDR3) amino acid sequence that exactly matches a known anti-EBV TCR CDR3 sequence; and predicting a higher OS probability if said anti-EBV TCR CDR3 amino acid sequence is present. In some examples, the TCR CDR3 sequences are TRA or TRB CDR3 sequences. In some examples, the anti-EBV TCR CDR3 sequences are identified from the VDJdb database. Some examples, further comprise quantifying chemical complementarity between the identified TCR CDR3 sequences and known EBV epitopes. The quantifying step utilizes hydrophobic, electrostatic, or combined chemical complementarity scores (CSs). In some examples, the anti-EBV TCR CDR3 amino acid sequences correspond to sequences known to bind EBV epitopes identified from immune epitope databases.

In one example, disclosed herein is a method of stratifying subjects with EBV-positive Burkitt lymphoma (BL) for treatment decisions, comprising: obtaining tumor RNA from a subject diagnosed with EBV-positive BL; sequencing said tumor RNA to identify T cell receptor (TCR) CDR3 amino acid sequences expressed by tumor-infiltrating lymphocytes; determining the presence or absence of matches between identified TCR CDR3 sequences and known anti-EBV TCR CDR3 amino acid sequences; and classifying the subject as having an increased likelihood of beneficial clinical outcomes based on the presence of at least one anti-EBV TCR CDR3 amino acid sequence. In some examples, the TCR CDR3 sequencing is performed by RNAseq. Some examples, further comprise determining a chemical complementarity score (CS) between identified TCR CDR3 sequences and EBV epitopes, wherein a higher CS indicates a higher likelihood of improved OS.

In one example, disclosed herein is a therapeutic method for improving overall survival (OS) of a subject diagnosed with EBV-positive Burkitt lymphoma (BL), comprising: identifying T cells expressing TCR CDR3 sequences reactive to EBV antigens from the subject or a donor; expanding said T cells ex vivo in the presence of one or more EBV-specific epitopes; and administering the expanded T cells to the subject, thereby eliciting an enhanced adaptive immune response to EBV and improving OS. In some examples, the EBV-specific epitopes comprise peptides derived from EBV nuclear antigen 3 (EBNA3). In some examples, the expanded T cells comprise autologous T cells. In some examples, the expanded T cells comprise donor-derived allogeneic T cells matched at major histocompatibility complex (MHC) loci. In some examples, the administration of expanded T cells is performed in combination with chemotherapy or immunotherapy.

In one example, disclosed herein is a system for immune profiling and survival prediction, comprising: a database of viral epitopes; a database of known antigen-specific TCRs; a processing module configured to receive TCR sequence data from a subject sample; a sequence-matching module for identifying exact matches between subject-derived TCRs and known TCRs; a chemical complementarity scoring module for calculating scores between subject-derived TCRs and viral epitopes; and a survival prediction module configured to stratify the subject based on matching results and/or complementarity scores. In some examples, the system is implemented as a web-based platform configured to receive TCR and epitope input data and return survival stratification results. In some examples, the chemical complementarity scoring module includes a Combo CS integrating both hydrophobic and electrostatic components.

In one example, disclosed herein is a method for determining subject prognosis based on T-cell receptor (TCR) sequences, comprising: obtaining a set of TCR CDR3 amino acid sequences from a subject sample; comparing the obtained TCR CDR3 sequences to a database of known antigen-specific TCR sequences; identifying exact amino acid sequence matches between the subject's TCR CDR3s and TCR sequences known to be reactive to Epstein-Barr virus (EBV) antigens; and stratifying the subject based on the presence or absence of exact matches to known EBV-reactive TCR sequences to determine an association with survival outcome. In some examples, the database of known antigen-specific TCR sequences comprises the VDJdb database.

In one example, disclosed herein is a method for predicting subject survival by assessing chemical complementarity between subject-derived TCR sequences and viral epitopes, comprising: obtaining TCR CDR3 amino acid sequences from a subject sample; obtaining a set of viral epitope amino acid sequences; calculating a chemical complementarity score (CS) between the TCR sequences and epitope sequences, the CS comprising at least one of: a hydrophobic interaction score, an electrostatic interaction score, or a combination thereof determining a maximal complementarity score for each subject; and stratifying the subject into a high or low complementarity group based on the maximal CS value to associate with subject survival outcome. In some examples, the viral epitope sequences are obtained from the Immune Epitope Database (IEDB). In some examples, the complementarity score is computed using an algorithm incorporating side-chain physicochemical properties of amino acids. In some examples, the survival prediction is generated using Kaplan-Meier analysis and/or Cox proportional hazards modeling.

### Methods of Immune Profiling Using IGH Recombination Reads

The current invention provides a method for mining IGH recombination reads from RNAseq files, identifying CDR3 regions, evaluating their chemical complementarity with known viral epitopes (especially from EBV), and predicting survival outcomes based on these scores. The invention includes: algorithms to extract IGH recombination reads from RNAseq data, scoring modules that assess chemical complementarity between IGH CDR3s and viral epitopes using hydrophobic and electrostatic models, stratification systems dividing patients into percentile groups based on maximum complementarity scores, use of Kaplan-Meier (KM) and Cox regression analyses for statistical validation, and integration with publicly available cancer genomics and clinical datasets (e.g., TCGA, CPTAC, CGCI). The invention is applicable across multiple cancer types and is validated against independent datasets and algorithms.

The method begins by receiving RNAseq files from tumor samples. An algorithm is applied to extract IGH recombination reads, which include variable (V), diversity (D), and joining (J) gene segments. The reads are translated into amino acid sequences, and the CDR3 region is identified as the segment lying between conserved sequence motifs.

CDR3 sequences are aggregated and sorted by frequency using tools such as Excel COUNTIF or programmatic scripts. Approximately the top 2,000 most frequent CDR3s are selected, with allowance for tie values.

Epitope sequences are retrieved from the Immune Epitope Database (IEDB), limited to EBV B cell assay validated linear epitopes. The top 100 epitopes are selected based on literature reference counts.

Each CDR3-epitope pair is aligned using a sliding window technique. A chemical complementarity score (CS) is calculated using one or more scoring functions: Hydrophobic CS measures hydrophobic interaction compatibility; Electrostatic CS measures charge-based residue complementarity; and Combo CS integrates score combining hydrophobic and electrostatic metrics. The maximum CS value per patient is used for stratification.

Patients are divided into “upper” and “lower” 50th percentile groups based on the maximum CS value. These groups are compared using: Kaplan-Meier survival curves, Log rank tests, and Cox proportional hazards models. The survival endpoints include overall survival (OS), disease-specific survival (DSS), disease-free survival (DFS), and progression-free survival (PFS).

The approach is validated across datasets including TCGA-OV, CPTAC-OV, CGCI-BLGSP, and NCICCR-DLBCL.

In one example, disclosed herein is method for identifying immunoglobulin heavy chain (IGH) CDR3 sequences from RNA sequencing data, comprising: receiving RNAseq files from a tumor tissue sample of a subject; applying a computational algorithm to extract IGH recombination reads from the RNAseq data; translating said recombination reads into amino acid sequences; identifying complementarity-determining region 3 (CDR3) sequences within the translated IGH sequences; and storing said IGH CDR3 sequences in association with a patient or case identifier. In some examples, the algorithm for extracting IGH recombination reads emphasizes matching V and J gene segment identities and determines whether a sequence is productive or unproductive.

In one example, disclosed herein is method for determining the overall survival in an EBV-positive cancer subject, comprising: obtaining IGH CDR3 amino acid sequences from a tumor sample of the subject; calculating chemical complementarity scores (CSs) between said IGH CDR3 sequences and a predefined set of EBV-derived epitope sequences; selecting a maximum CS for each subject; stratifying the subjects into an upper 50th percentile group and a lower 50th percentile group based on said maximum CS; and associating the percentile groupings with one or more clinical survival outcomes, including overall survival (OS), disease-specific survival (DSS), disease-free survival (DFS), or progression-free survival (PFS). In some examples, the clinical survival data is accessed from public repositories selected from the group consisting of: cbioportal.org and the Genomic Data Commons (GDC) portal. Some examples, further comprise generating Kaplan-Meier survival plots and Cox proportional hazards models to determine statistically significant survival distinctions between percentile groups. In some examples, the complementarity scoring is verified by an Excel pivot table to confirm stratification groupings based on maximum CS values. In some examples, the stratification is validated using a second IGH CDR3 dataset obtained using a distinct IGH mining algorithm. In some examples, the IGH-epitope interactions are contrasted against control non-EBV epitopes to determine statistical significance of EBV epitope associations. In some examples, the tumor staging or tumor grade is compared between upper and lower percentile groups using a Chi-squared statistical test. In some examples, the gender-based stratification is performed by comparing the proportion of male and female patients in each CS-based percentile group using a two-proportion test.

Also, disclosed herein is a method for evaluating chemical complementarity between IGH CDR3 amino acid sequences and viral epitopes, comprising: selecting a set of frequently occurring IGH CDR3 amino acid sequences from tumor-derived RNAseq data; retrieving a plurality of epitope sequences derived from Epstein-Barr Virus (EBV) from a reference epitope database; performing pairwise sequence alignment using a sliding window algorithm; calculating a chemical complementarity score for each IGH CDR3 epitope pair based on one or more physicochemical properties; and ranking or grouping the IGH CDR3-epitope interactions based on said chemical complementarity score. In some examples, the chemical complementarity score comprises a hydrophobic score computed by comparing hydrophobic residue profiles between aligned sequences. In some examples, the epitope database comprises B-cell assay-derived EBV epitopes obtained from the Immune Epitope Database (IEDB), ranked by the number of literature references.

### Adoptive Immune Therapies

A TCR is described as a heterodimeric cell surface protein of the immunoglobulin super-family, which may associate with invariant proteins of the CD3 complex that are involved in mediating signal transduction. TCRs can exist in αβ and γδ forms, which are structurally similar but may possess distinct anatomical locations and potentially different functions. The alpha and beta chains of a native heterodimeric αβTCR may be transmembrane proteins, each potentially comprising two extracellular domains: a membrane-proximal constant domain and a membrane-distal variable domain. Each of the constant and variable domains might include an intra-chain disulfide bond.

The variable region of each TCR chain may comprise variable and joining segments, and in the case of the beta chain, also a diversity segment. Each variable region could comprise three CDRs (Complementarity Determining Regions), which are highly polymorphic loops embedded in a framework sequence, one being the hypervariable region named CDR3. Several types of alpha chain variable (Vα) regions and several types of beta chain variable (Vβ) regions may be distinguished by their framework, CDR1 and CDR2 sequences, and a partly defined CDR3 sequence. Unique TRAV or TRBV numbers may be given to Vα or Vβ regions by IMGT nomenclature. TCR specificity for the recognized epitopes is believed to be mainly determined by the CDR3 regions (Danska et al., 1990; Garcia et al., 2005).

The use of adoptive TCR gene therapy may enable equipping a subject's own T cells with desired specificities and the generation of sufficient numbers of activated, non-exhausted T cells in a short time. A TCR may be transduced into all T cells or into T-cell subsets such as CD8+ T cells, central memory T cells, or T cells with stem cell characteristics, potentially ensuring better persistence and function upon transfer. TCR-engineered T cells may then be infused into subjects, for example, those with cancer who might have been rendered lymphopenic through chemotherapy or irradiation, thereby inducing homeostatic expansion that may enhance engraftment and long-term persistence of transferred T cells and could be associated with higher cure rates.

TCR-based adoptive T cell therapy is understood to rely on classical TCR recognition of processed epitopes of antigens presented by certain MHC molecules. Thus, a T cell expressing a specific TCR for a given epitope and MHC combination might only be useful for treating subjects expressing the corresponding MHC.

Epstein-Barr virus (EBV), a human herpesvirus, is believed to infect approximately 90% of the global population. In healthy individuals, diseases caused by EBV may be cleared by immune cells, particularly T cells. EBV-positive diseases may include infectious mononucleosis and a range of non-malignant, premalignant, and malignant EBV-positive lymphoproliferative diseases such as post-transplant lymphoproliferative disorder, Burkitt lymphoma, hemophagocytic lymphohistiocytosis, Hodgkin and non-Hodgkin lymphomas; non-lymphoid malignancies such as gastric cancer, lung cancer, and nasopharyngeal carcinoma; and conditions associated with human immunodeficiency virus such as hairy leukoplakia and central nervous system lymphomas. The virus might also be associated with childhood disorders such as Alice in Wonderland Syndrome and acute cerebellar ataxia and, based on some evidence, an increased risk of developing certain autoimmune diseases. Approximately 200,000 cancer cases per year may be attributable to EBV.

Most EBV-positive cancers are thought to express only a limited number of EBV-specific antigens such as latent membrane proteins (LMP1, LMP2A) and nuclear proteins (EBNA1, EBNA3C). These antigens may represent interesting targets for TCR-based immunotherapies such as TCR gene therapy or adoptive T cell therapy for EBV-positive diseases, including post-transplant lymphoproliferative disorder or cancer (Orentas et al., 2001; Jurgens et al., 2006; Hart et al., 2008; Simpson et al., 2011; Yang et al., 2011; Zheng et al., 2015; Cho et al., 2018; WO 2015/022520 A1; WO 2011/039508 A2). However, most T cell-based immunotherapies targeting EBV-positive malignancies have been using natural EBV-specific T cells generated from third party donors or patients, where T cells have been expanded using EBV lymphoblastoid cell lines (LCL) or EBV peptide pools. Adoptive T cell therapies using EBV-specific TCR-engineered T cells have not been tested in clinical trials. TCR-engineered T cells have several advantages compared to natural EBV-specific T cells: 1) Efficacy: The introduced TCR is a pre-defined receptor with high affinity to EBV-positive tumor cells. Growing natural T cells from patient blood relies on the presence of EBV-specific T cells to grow out. However, a patient may lack effective T cells that can be expanded. 2) Feasibility: The success rate of manufacturing engineered T cells is above 95%, while procedures to grow natural T cells have success rates below 70%. 3) Costs: Vein-to-vein time is reduced to less than 21 days in engineered T cell processes compared to more than 40 days for expanding natural T cells.

In some examples, the nucleic acid might be a viral vector or a non-viral vector such as a transposon, a vector suitable for CRISPR/Cas-based recombination, or a plasmid suitable for in vitro RNA transcription. Preferably, the nucleic acid may be a vector. Suitable vectors might be designed for propagation and expansion, expression, or both, such as plasmids and viruses. The vector may be an expression vector suitable for use in a host cell, which could be a human T cell or a precursor thereof, preferably a CD8+ T cell such as a central-memory, effector-memory, stem-like, or effector T cell. The vector might be viral, e.g., a retroviral vector like MP71 (Engels et al., 2003).

The expression vector may include regulatory sequences for transcription and translation initiation and termination, specific to the host cell type (e.g., bacterium, fungus, plant, or animal), where expression of the nucleic acid might occur, typically in human CD8+ T cells. The vector could include one or more marker genes for selecting transduced or transfected cells. The promoter might be a heterologous promoter (e.g., LTR) suitable for TCR expression in human T cells. Expression could be transient or stable, constitutive or inducible.

The host cell may be eukaryotic (e.g., plant, animal, fungi) or prokaryotic (e.g., bacteria). Preferably, the host cell is a mammalian, more preferably, a human cell. The host cell might be a cultured or primary cell, adherent or suspended. For producing recombinant TCR proteins, the host cell is preferably a human T cell or precursor, which might be isolated from PBMCs. T cells may be sourced from blood, bone marrow, lymph nodes, thymus, or other tissues. These may include tumor-infiltrating lymphocytes (TILs), central memory, effector, or naïve T cells.

Preferably, the T cell is a human CD8+ T cell genetically engineered to express a TCR construct encoded by a nucleic acid under a heterologous promoter. The host cell might also express two or more TCR constructs, for example, two single-chain constructs joined by linkers to avoid mispairing.

In some instances, the subject may have an EBV-positive cancer such as Hodgkin or non-Hodgkin lymphoma, Burkitt lymphoma, hemophagocytic lymphohistiocytosis, nasopharyngeal carcinoma, or others. Preferably, the cancer is a type II (e.g., Hodgkin lymphoma) or type III malignancy (e.g., post-transplant lymphoproliferative disorder), which may express high levels of LMP1, LMP2A, and possibly EBNA2C.

Targeted cancer cells may express the antigen(s) from which the recognized epitope is derived, preferably on most cells. The subject is typically a mammal, preferably a human.

The invention may include a method of treating cancer or an infectious disease, such as an EBV-positive disease, by administering a pharmaceutical composition or kit to a subject in need thereof. Such compositions might be used with other anti-cancer agents, including additional TCR-engineered T cells, checkpoint inhibitors, antibodies, small molecules, or other reagents.

A preferred medicinal use involves immune therapy, particularly adoptive T cell therapy. T cells may be autologous and transduced in vitro with a nucleic acid of the invention, or the nucleic acid may be administered directly to transduce T cells in vivo.

Protein TCR constructs might also be used diagnostically, for example, to determine whether a subject presents the epitope recognized by the construct. In such cases, adoptive T cell therapy may be initiated.

Additionally, a method of preparing a host cell might involve introducing an expression vector encoding a TCR construct into a suitable host cell, preferably a CD8+ T cell from the subject.

### Compositions

The present disclosure relates to compositions and methods for preventing or treating Epstein-Barr virus (EBV)-positive cancers. In certain examples, the therapy comprises adoptive or engineered immune effector cells, including chimeric antigen receptor (CAR) T or NK cells specific for EBV antigens such as latent membrane protein 2A (LMP2A), latent membrane protein 1 (LMP1), or the viral envelope glycoprotein gp350 (for example constructs using a 72A1-derived single-chain variable fragment, scFv), as well as EBNA3A/3B/3C-specific TCR-transduced T cells. Additional examples include third-party EBV-specific cytotoxic T lymphocytes (CTLs), exemplified by tabelecleucel (tab-cel, ATA129), and multi-virus products that include EBV reactivity such as posoleucel (ALVR105). Engineered cells may further be edited by CRISPR or TALEN to knock out inhibitory receptors (for example PD-1) or endogenous TCRs, or configured as CAR NK cells (for example NK-92 or primary NK cells bearing gp350- or LMP1-binding domains), including trispecific or cytokine-armed versions (for example IL-15 expressing products). In other examples, the invention provides monoclonal antibodies that neutralize or deplete EBV-infected cells, including but not limited to rituximab (anti-CD20), 72A1 (anti-gp350), AMMO1 (anti-gH/gL), E1D1 (anti-gp42), and S12 (anti-LMP1). The disclosure further contemplates isolated scFv fragments derived from such antibodies, for example 72A1 scFv, E1D1 scFv, S12 scFv, and panels of LMP2A-specific scFvs, which can function as stand-alone inhibitors, targeting moieties in multispecific formats, or antigen-recognition domains in CAR constructs. Additional protein formats include EBV-targeted bispecific T cell engagers (BiTEs) or dual-affinity retargeting molecules (DARTs), such as gp350×CD3, LMP1×CD3, or LMP2A×CD3 constructs, as well as soluble gp350 or gp42 decoys that sequester host receptors. Certain examples combine direct antivirals (for example acyclovir, ganciclovir, valganciclovir, cidofovir, foscarnet, or maribavir) with pharmacologic induction of the EBV lytic cycle (“kick and kill”), using histone deacetylase inhibitors (for example vorinostat, romidepsin, valproic acid), proteasome inhibitors (for example bortezomib), cytotoxic agents such as gemcitabine, and DNA methyltransferase inhibitors (for example decitabine, azacitidine), optionally including earlier butyrate derivatives. In further examples, EBV genomes or transcripts are directly targeted by gene-editing or gene-silencing technologies, including CRISPR/Cas systems (Cas9 or Cas12a) directed to EBNA1, OriP, or LMP1, zinc finger nucleases, TALENs, antisense oligonucleotides, siRNAs, morpholinos, locked nucleic acids, or dCas9-KRAB fusions to epigenetically silence latent promoters (for example Cp, Wp, or Qp). Vaccine approaches are also described, including recombinant gp350 proteins or virus-like particles, nanoparticle-display immunogens of gH/gL or gB (for example ferritin or I53-50 scaffolds), DNA or viral-vector vaccines encoding latency antigens such as EBNA1, LMP2, or BARF1, mRNA vaccines encoding latency antigens, and multi-epitope peptide formulations emphasizing EBNA3 family epitopes. Indirect anti-EBV oncologic strategies that modulate host signaling pathways are encompassed, including NF-kappaB pathway inhibitors, JAK/STAT inhibitors such as ruxolitinib or STAT3 inhibitors (for example napabucasin), BCL-2 inhibition (for example venetoclax), and immune checkpoint blockade (for example nivolumab, pembrolizumab, toripalimab) to restore EBV-specific immunity. Miscellaneous biologic formats such as nanobodies targeting LMP1 or gp350, aptamers, and designed ankyrin repeat proteins (DARPins) are also within the scope of the disclosure. The foregoing examples are provided to illustrate the breadth of anti-EBV modalities contemplated and are not intended to limit the scope of the claims, which may encompass any combination, formulation, dosing schedule, or manufacturing method that achieves reduction, elimination, or prevention of EBV-positive cancers.

In one example, disclosed herein is a therapeutic composition comprising: T cells expressing at least one anti-EBV TCR CDR3 amino acid sequence known to specifically recognize EBV antigens; and a pharmaceutically acceptable carrier suitable for administration to subjects with EBV-positive BL. In some examples, the T cells are autologous T cells expanded ex vivo. In some examples, the T cells have enhanced chemical complementarity scores (CSs) with at least one EBV epitope, relative to a reference set of T cells without anti-EBV specificity. In some examples, expanding said T cells ex vivo comprises culturing the T cells with antigen-presenting cells pulsed with EBV epitopes. Some examples, further comprise genetically engineering T cells to express TCRs specific for EBV epitopes prior to administering to the subject.

In one example, disclosed herein is a nucleic acid encoding a TCR alpha chain construct (TRA) and/or a TCR beta chain construct (TRB) of a TCR construct complementary to an Epstein-Barr-virus (EBV) epitope, wherein the epitope has the sequence of SEQ ID NO: 4, SEQ ID NO: 5 or SEQ ID NO: 6.

In one example, disclosed herein is a host cell comprising a nucleic acid encoding a TCR alpha chain construct (TRA) and/or a TCR beta chain construct (TRB) of a TCR construct complementary to an Epstein-Barr-virus (EBV) epitope, wherein the epitope has the sequence of SEQ ID NO: 4, SEQ ID NO: 5 or SEQ ID NO: 6, and wherein the host cell preferably is a human CD8+ T cell.

In one example, disclosed herein is a pharmaceutical composition comprising: a host cell, wherein the host cell comprises a nucleic acid acid encoding a TCR alpha chain construct (TRA) and/or a TCR beta chain construct (TRB) of a TCR construct complementary to an Epstein-Barr-virus (EBV) epitope, wherein the epitope has the sequence of SEQ ID NO: 4, SEQ ID NO: 5 or SEQ ID NO: 6; and a pharmaceutically acceptable carrier, an adjuvant or a combination thereof. In some examples, the pharmaceutically acceptable carrier comprises a nanoparticle or a liposome. In some examples, the adjuvant is alum. In some examples, the pharmaceutical composition induces an adaptive immune response against EBV epitopes in cancer subjects. In some examples, the EBV epitopes are selected from LMP2A, LMP1, EBNA1 or EBNA3C.

### Treatments

In one example, disclosed herein is a method of treating an EBV-positive cancer in a subject, comprising:
a) obtaining a biological sample from the subject, wherein the biological sample comprises tumor-infiltrating lymphocytes (TIL);
b) extracting complementarity determining region 3 (CDR3) amino acid (AA) sequences from the TIL, thereby obtaining extracted TIL CDR3 AA sequences;
c) identifying an exact match of extracted TIL CDR3 AA sequences to known anti-EBV CDR3 AA sequences from the biological sample; thereby obtaining an exact match anti-EBV TIL CDR3 AA sequence; and
d) administering a pharmaceutically effective amount of an anti-EBV therapeutic to the subject with a presence of the exact match anti-EBV TIL CDR3 AA sequence,

wherein the presence of the exact match anti-EBV TIL CDR3 AA sequence in the sample is correlated to better overall survival.

In one example, disclosed herein is a method of treating EBV-positive cancer in a subject, comprising:
a) obtaining a biological sample from the subject, wherein the biological sample comprises tumor-infiltrating lymphocytes (TIL);
b) extracting complementarity determining region 3 (CDR3) amino acid (AA) sequences from the TIL, thereby obtaining extracted TIL CDR3 AA sequences;
c) obtaining a chemical complementarity score (CS) by interacting known EBV epitopes AA sequences to the extracted TIL CDR3 AA sequences; and
d) administering a pharmaceutically effective amount of an anti-EBV therapeutic to the subject with a high CS, wherein a high CS is correlated to higher overall survival.

In some examples, the CS is calculated using hydrophobic interactions, electrostatic interactions, or a combination thereof.

In some examples, the biological sample comprises blood or tumor biopsy.

In some examples, the TILs comprise B cells or T cells.

In some examples, the T cell comprises T cell receptor (TCR), wherein the TCR comprises an alpha chain or a beta chain.

In some examples, the B cell comprises B cell receptor (BCR), wherein the BCR comprises two heavy chains (IGH) and two light chains (IGL).

In some examples, the anti-EBV therapeutic comprises an anti-viral therapeutic against EBV epitopes, an adoptive T cell therapy targeting EBV epitopes, a single-chain variable fragment (scFv) targeting EBV epitopes, or an anti-EBV antibody.

In some examples, the anti-EBV therapeutic is administered intravenously, intramuscularly, intraperitoneally, intradermally, or subcutaneously to the subject.

In some examples, the EBV-positive cancer comprises nasopharyngeal carcinoma (NPC), Hodgkin lymphoma, Burkitt lymphoma, post-transplant lymphoproliferative disorder (PTLD), gastric carcinoma, or ovarian cancer.

In some examples, disclosed herein is a method for treating EBV-cancer in a subject, comprising:
determining the chemical complementarity score (CS) between subject-derived TRA or TRB TCR CDR3 sequences and EBV epitopes, wherein said CS is computed based on hydrophobic, electrostatic, or combined physicochemical interactions; and
administering an immune checkpoint inhibitor, EBV-specific vaccine, or adoptive TCR-based T-cell therapy to the subject identified as having a high CS indicative of effective immune recognition; or
administering engineered TCR or CAR-T cell therapy, EBV-targeted antibody therapy, intensive chemotherapy, or targeted molecular therapy to the subject identified as having a low CS indicative of impaired immune recognition.

In some examples, the immune checkpoint inhibitor is selected from anti-PD-1 or anti-PD-L1 antibodies.

In some examples, the high CS is defined as a complementarity score in the upper 50th percentile compared to a reference population of cancer subjects.

In some examples, a low CS is defined as a complementarity score in the lower 50th percentile compared to a reference population of cancer subjects.

In some examples, the subject as having a shorter overall survival comprises the subject having an overall survival of about 1 month or less, about 2 months or less, about 4 months or less, about 6 months or less, about 8 months or less, about 10 months or less, about 12 months or less, about 14 months or less, about 16 months or less, about 18 months or less, about 20 months or less, about 22 months or less, about 25 months or less, or about 30 months or less, about 35 months or less, about 40 months or less, about 45 months or less, about 50 months or less, about 55 months or less, about 60 months or less, about 65 months or less, about 70 months or less, about 75 months or less, about 80 months or less, about 85 months or less, about 90 months or less, about 95 months or less, about 100 months or less, about 150 months or less, about 200 months or less, or about 250 months or less.

In some examples, the subject is a human. In some examples, the human has or is suspected of having a cancer. In some examples, the cancer is selected from the group consisting of neuroblastoma, low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

Accordingly, in some examples, a complementarity score that is less than 0 denotes the subject as having a longer overall survival. Accordingly, in some examples, the subject is administered with a therapeutically effective amount of an anti-cancer agent if the complementarity score is more than 0.

In some examples, the anti-cancer agent is selected from the group consisting of anti cordycepin, fenretinide, Zyclara, vemurafenib (Zelboraf®), dabrafenib (Tafinlar™), encorafenib (Braftovi™), pembrolizumab (Keytruda), nivolumab (Opdivo), Anthracyclines, Taxanes, 5-fluorouracil (5-FU), Cyclophosphamide (Cytoxan), Carboplatin (Paraplatin), cisplatin, carboplatin, Vinorelbine (Navelbine), Capecitabine (Xeloda), Gemcitabine (Gemzar), Ixabepilone (Ixempra), Eribulin (Halaven), Fulvestrant (Faslodex), Letrozole (Femara), Anastrozole (Arimidex), exemestane (Aromasin), Trastuzumab (Herceptin), Pertuzumab (Perjeta), Ado-trastuzumab emtansine, Lapatinib (Tykerb), Neratinib (Nerlynx), Everolimus (Afinitor), Olaparib (Lynparza), talazoparib (Talzenna), Alpelisib (Piqray), Atezolizumab (Tecentriq), Paclitaxel (Taxol), Albumin-bound paclitaxel (nab-paclitaxel, Abraxane), Docetaxel (Taxotere), Etoposide (VP-16), Pemetrexed (Alimta), Bevacizumab (Avastin), Ramucirumab (Cyramza), ifosfamide (Ifex™), irinotecan (Camptosar™), mitomycin, doxorubicin (Adriamycin), methotrexate, vinblastine (CMV), durvalumab (Imfinzi™), avelumab (Bavencio™), Erdafitinib (Balversa), dacarbazine (DTIC), epirubicin, temozolomide (Temodar™), gemcitabine (Gemzar™), trabectedin (Yondelis™), and Pazopanib (Votrient).

As would be apparent, the sequencing may be done using a next generation sequencing platform, e.g., Illumina's reversible terminator method, Roche's pyrosequencing method, Life Technologies' sequencing by ligation (the SOLiD platform) or Life Technologies' Ion Torrent platform, etc. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps. In other examples, the sequencing may be done using nanopore sequencing (e.g. as described in Soni et al Clin Chem 53: 1996-2001 2007, or as described by Oxford Nanopore Technologies).

### EXAMPLES

The following examples are set forth below to illustrate the compounds, systems, methods, and results according to the disclosed subject matter. These examples are not intended to be inclusive of all examples of the subject matter disclosed herein, but rather to illustrate representative methods and results. These examples are not intended to exclude equivalents and variations of the present invention which are apparent to one skilled in the art.

### Example 1. Detection of Anti-EBV TCR CDR3s Associated with Better Outcomes for EBV-Positive, Ugandan Cases of Burkitt Lymphoma

Burkitt lymphoma (BL) was first identified in Africa by Denis Burkitt in the mid-1900s. It is an aggressive form of non-Hodgkin lymphoma (NHL). BL can be classified into three forms: endemic, sporadic, or immunodeficiency related. In sub-Saharan Africa, BL is considered endemic and serves as the primary focus of this study. In this region, all patients diagnosed with endemic BL are found to have Epstein-Barr virus (EBV) infection. While several hypotheses have been proposed to explain the link between EBV and BL, the precise mechanism remains unclear.

In sub-Saharan Africa, BL shows a strong association with EBV. Though this association has been widely reported, the impact of the adaptive immune response, particularly involving anti-EBV T-cell activity in this population, remains underexplored. An analysis of T-cell receptor (TCR) complementarity-determining region 3 (CDR3) sequences from EBV-positive BL tumor samples from Uganda showed that the presence of anti-EBV CDR3s was associated with improved overall survival (OS). Additionally, chemical complementarity assessments revealed higher TCR-epitope complementarity in samples that contained anti-EBV CDR3 amino acid (AA) sequence matches.

In high-resource settings, BL treatment using combination chemo-immunotherapies has achieved cure rates of up to 95%. However, such treatments are generally unavailable in Africa due to limited resources and delayed diagnosis and management, resulting in over 50% of affected children not surviving past childhood.

EBV-positive tumors have shown worse outcomes in contexts where the virus can evade or suppress the immune system. Conversely, the presence of tumor-infiltrating lymphocytes, indicating an active immune response to EBV, has been associated with improved survival in certain cancers, such as gastric cancer. Despite experimental therapies like chemotherapy and T-cell therapy targeting EBV-positive tumors, improved management of EBV-positive BL remains a challenge. To date, no correlation has been definitively established between quantifiable immune responses to EBV and outcomes in EBV-related BL. Identifying such correlations could lead to novel therapies and improve outcomes, particularly in resource-limited regions.

To better understand the immune response in EBV-associated BL, TRA and TRB recombination reads were extracted from RNAseq data of BL tumors, using established software tools. These sequences were matched against previously identified anti-EBV TCR CDR3 AA sequences. Findings revealed that BL patients with anti-EBV TCR CDR3s exhibited better survival outcomes.

### Methods

Recovery of Adaptive Immune Receptor (IR) Recombinations from Cancer Genomic Characterization Initiative Burkitt Lymphoma Genome Sequencing Project (CGCI-BLGSP) Samples.

The algorithms and software for extracting the TRA and TRB recombination reads from the CGCI-BLGSP RNAseq files have been extensively described. The access to the CGCI-BLGSP RNAseq files (phs000235) was via the National Institutes of Health (NIH) database of genotypes and phenotypes project approval number 31203. The latest iteration of the software for extracting adaptive IR recombination reads from genomics files is freely available at GitHub. The TRA and TRB recombination reads were taken only from primary tumor samples.

### Accessing the Anti-EBV TCR CDR3s at VDJdb.

The VDJdb website antigen browser was queried for human TRA and TRB CDR3 AA sequences known to represent TCR reactivity with EBV antigens. The resulting 11370 anti-EBV TCR CDR3s, were used to determine which CGCI-BLGSP cases had TCR CDR3s that were exact AA sequence matches to the VDJdb, anti-EBV TCR CDR3s AA sequences.

### Immune Epitope Database EBV Antigens.

The Immune Epitope Database (IEDB) was queried for known EBV epitopes. Three queries were used to produce epitope collections analyzed in Results. Filters applied for the query of the first IEDB search were as follows: (a) epitope structure→linear sequence; (b) antigen→Epstein-Barr nuclear antigen 3 [P12977] (Human herpesvirus 4 (Epstein Barr virus)); (c) include positive assays; (d) no B-cell assays, no MHC assays; (e) Homo sapiens host. The resulting 29 EBV epitopes from these IEDB search parameters, all from T cell assays, were compiled into a CSV file for input at adaptive match. Filters applied for the query of the second IEDB search were as follows: (a) epitope structure→linear sequence; (b) organism→Human herpesvirus 4 (Epstein Barr virus), Human herpesvirus 4 strain M81 (Epstein-Barr virus (strain M81)), Human herpesvirus 4 type 1 (Epstein-Barr virus type 1), Human herpesvirus 4 type 2 (Epstein-Barr virus type 2), Human herpesvirus 4 strain CAO (Epstein-Barr virus (strain CAO)), Human herpesvirus 4 strain RAJI (Epstein-Barr virus (strain RAJI)), Human herpesvirus 4 strain B95-8 (Epstein-Barr virus (strain B95-8)); (c) include positive assays; (d) no B-cell assays, no MHC assays; (e) Homo sapiens host. This second search resulted in 644 EBV T-cell assay epitopes. Of these 644 epitopes, the EBV T-cell assay epitopes with 5 or more references were compiled for a subsequent analysis using the Adaptive Match web tool. Filters applied for the query of the third IEDB search were the same as described above for the second search of IEDB, except the epitopes represented B cell assays only. This third search resulted in 2556 EBV B-cell assay epitopes. Of these 2556 epitopes, the EBV B-cell assay epitopes with 2 or more references were compiled for a subsequent analysis using the Adaptive Match web tool. Excel macros were used to determine whether any epitopes were present in both the full EBV T-cell assay and B-cell assay epitope groups. Thirty epitopes were found to be precisely the same AA sequences for the T-cell and B-cell assay EBV epitope groups. These 30 epitopes were compiled for a subsequent analysis using the Adaptive Match web tool. Note that any epitopes in this overlapping group were excluded from individual T-cell assay and B-cell assay EBV epitope analyses as presented and detailed in the Results.

### Clinical and Survival Information Related to the CGCI-BLGSP Cases.

The Genomic Data Commons (GDC) database was accessed to obtain survival, clinical, and demographic information for the CGCI-BLGSP cases. Of all CGCI-BLGSP cases, only those cases of Ugandan origin were included in this report.

### Using Web Tool Adaptivematch.com.

The Adaptive Match web tool (adaptivematch.com) was used for the assessment of survival comparisons as well as for the output of chemical complementarity scores (CSs). Three different types of CSs, Hydrophobic (Hydro), Electrostatic, and Combo, based on previously indicated algorithms, are generated by matching CDR3 AA sequences to EBV, TCR epitopes (iedb.org). Note that the Combo CS is an integration of the Hydro and Electrostatic CSs. The survival data of each CGCI-BLGSP case were inputted with the case-associated CGCI-BLGSP TCR CDR3s and EBV epitopes derived from the IEDB as indicated above. The Adaptive Match web tool then returned Univariate Cox-regression p-values, KM p-values, and median survival times for the upper and lower 50th percentile case groups, with the upper and lower 50th percentile case groups based on the maximal CS value for each case.

### Use of R Studio.

The survival data from the GDC as detailed above were subsequently imported into R Studio to verify the output from adaptivematch.com and to create KM plots for analyses and figures. The R Studio application was used to create KM plots and censoring tables for several different comparisons throughout this report using the survminer package.

### Results

OS Distinctions for Ugandan CGCI-BLGSP Cases with TRA and TRB CDR3s with an Anti-EBV TCR CDR3 Match Versus Cases with No Anti-EBV TCR CDR3 Matches.

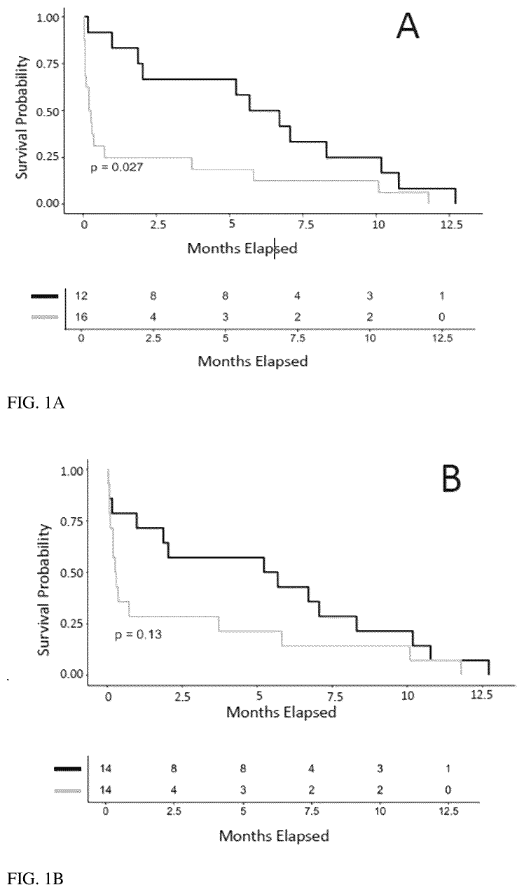

Following the recovery of TRA and TRB recombination reads from RNAseq files representing primary tumor samples from Ugandan CGCI-BLGSP cases (Methods), 62 cases were identified which had TRA or TRB CDR3s with at least one exact AA sequence match to the AA sequences representing the set of anti-EBV TRA and TRB CDR3s from VDJdb. Survival data obtained from the GDC were available for 28 of these 62 cases. A KM analysis comparing the CGCI-BLGSP Ugandan cases with a TRA CDR3 that exactly matched the AA sequence of an anti-EBV TRA CDR3, versus the CGCI-BLGSP Ugandan cases with productive TRA recombination reads and no anti-EBV TRA CDR3 matches, shows a significant difference in survival probability for these two groups of cases (FIG. 1A). Specifically, the Ugandan CGCI-BLGSP cases with an anti-EBV TRA CDR3 match represented an increased overall survival (OS) probability (FIG. 1A; log rank p=0.027). A second KM analysis indicated that CGCI-BLGSP Ugandan cases with a TRA or TRB, anti-EBV CDR3 match, versus the CGCI-BLGSP Ugandan cases with neither a TRA nor TRB anti-EBV CDR3 match, indicated a trend towards better survival probability for cases with the anti-EBV TCR CDR3 matches (FIG. 1B; log rank p=0.13).

### Quantifying the Chemical Complementarity of Ugandan, CGCI-BLGSP TRA and TRB CDR3s and EBV Epitopes.

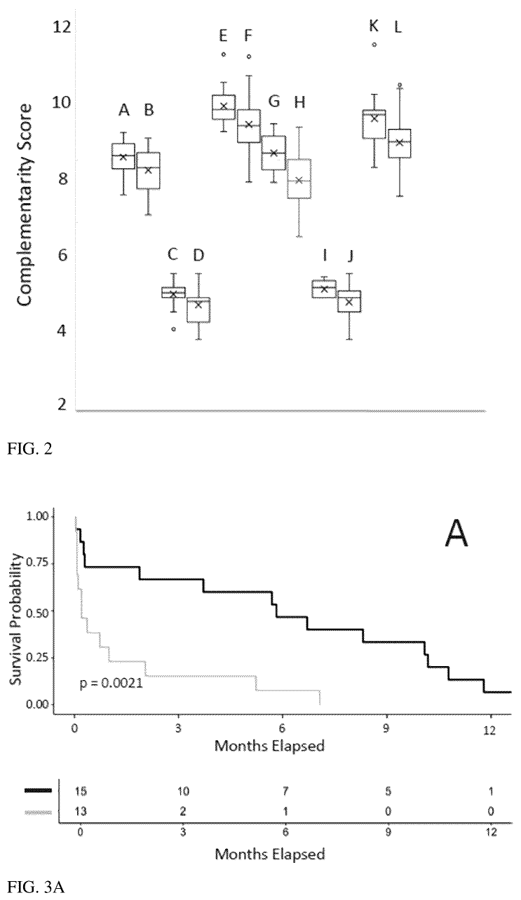

Chemical complementarity scores (CSs) for four different groups of CDR3s were compared, a comparison that was facilitated by the Adaptive Match web tool (Methods). The four groups of CDR3s were as follows: (a) the TRA CDR3s from Ugandan CGCI-BLGSP cases with at least one anti-EBV TRA CDR3 match (based on the TRA anti-EBV CDR3s from VDJdb); (b) the TRA CDR3s from Ugandan CGCI-BLGSP cases with no anti-EBV TRA CDR3 matches; (c) the TRB CDR3s from Ugandan CGCI-BLGSP cases with at least one anti-EBV TRB CDR3 match; and (d) the TRB CDR3s from Ugandan CGCI-BLGSP cases with no anti-EBV TRB CDR3 matches. The Ugandan CGCI-BLGSP TRA and TRB CDR3 collections representing these four groups, (a-d), respectively, were paired with 29 T-cell, EBV EBNA3 epitopes from IEDB (Methods) for the calculations of the CDR3-epitope CSs. That is, with 29 distinct T-cell epitopes in the IEDB, EBNA3 represented a comparatively credible T-cell antigen. Hydro, Electrostatic, and Combo CSs, respectively, were obtained for every CDR3-EBV epitope pair. The maximum Hydro, Electrostatic, and Combo CSs, respectively, for each Ugandan CGCI-BLGSP case were identified. The mean of the maximum CS values for each of the four groups indicated above (a-d) was then obtained, again keeping in mind that each of the Hydro, Electrostatic and Combo CSs values were assessed separately. Thus, the average of the maximum, Hydro CSs for TRA CDR3-EBV epitope pairs, when the TRA CDR3s were obtained from Ugandan CGCI-BLGSP cases with at least one anti-EBV TRA CDR3 match (group a, above), was 8.08; for cases with no anti-EBV TRA CDR3 matches (group b), the average of the maximum, Hydro CSs for TRA CDR3-EBV epitope pairs was 7.65 (FIG. 2; Table 1, p=0.0053). This same comparison was then made for the Electrostatic and Combo CSs, based on the TRA CDR3-EBV epitope pairs; and for the Hydro, Electrostatic, and Combo CSs, based on the TRB CDR3-EBV epitope pairs. In all cases, the mean of the maximum CSs represented by cases with TRA and TRB anti-EBV CDR3 matches, respectively, was higher than the corresponding CSs representing the cases with no anti-EBV TCR CDR3 matches (FIG. 2; Table 1).

TABLE 1
Student's t-test for comparison of the Hydro, Electrostatic,
and Combo CSs of 29 EBV EBNA3 epitopes when paired with Ugandan
TRA or TRB CDR3s from case sets representing a match to anti-
EBV CDR3s versus cases with no match to anti-EBV CDR3s.

T-test p-value

| Type of CS | TRA | TRB |
|---|---|---|
| Hydro CS | 0.0053378 | 0.00146851 |
| Electrostatic CS | 0.00596698 | 0.00100957 |
| Combo CS | 0.00202497 | 0.0418221 |

Statistical analyses for data represented by FIG. 2.

### Survival Analysis of Cases Representing Upper and Lower 50th Percentile of CGCI-BLGSP TCR CDR3-T-Cell Assay EBV Epitope CS Groups.

To investigate potential survival probability differences for cases representing the upper and lower 50th percentile CS groups, individual Ugandan CGCI-BLGSP case TRA and TRB CDR3s were combined for an assessment of TCR CDR3-EBV epitope CSs for each case. Survival analyses for Ugandan CGCI-BLGSP cases with TCR CDR3s and with associated survival data from GDC were performed. Specifically, the CSs for the TCR CDR3s and 44 highly referenced EBV T-cell assay epitopes indicated five epitopes that represented unusually high OS probability distinctions based on the Electrostatic CS calculations. These OS probability distinctions were based on the assignment of the BL cases to either the upper or lower 50th percentile group based on the maximum CS for a given case. The OS probability distinctions of the Ugandan CGCI-BLGSP cases based on Electrostatic CSs between TCR CDR3s and the above indicated, five T-cell EBV epitopes all represented a log rank p-value of less than 0.03 (Table 2). The five EBV epitopes representing these survival probability distinctions had the following IEDB designations: IEDB-27992, IEDB-53128, IEDB-29466, IEDB-50298, and IEDB-5316 (Table 2).

TABLE 2
KM analyses output based on Electrostatic
CSs of Ugandan CGCI-BLGSP TRA and TRB CDR3s
matched with T-cell assay EBV epitopes.

| | | | Cases in the | Cases in the |
|---|---|---|---|---|
| | | | upper 50^{th} | lower 50^{th} |
| IEDB | Univariate | | percentile of | percentile of |
| epitope | Cox | KM log | Electrostatic | Electrostatic |
| number | regression | rank p- | CSs: median | CSs: median |
| designation | p-value | value | days of survival | days of survival |
| 27992 | 0.001 | 0.0291 | 159 | 6 |
| 53128 | 0.004 | 0.0003 | 177 | 6 |
| 29466 | 0.006 | 0.0004 | 177 | 5 |
| 50298 | 0.013 | 0.0166 | 177 | 9 |
| 5316 | 0.048 | 0.0001 | 177 | 5 |

Note,
the cases were assigned to the upper or lower 50^{th} percentile based on the maximum Electrostatic CS for the indicated epitope.

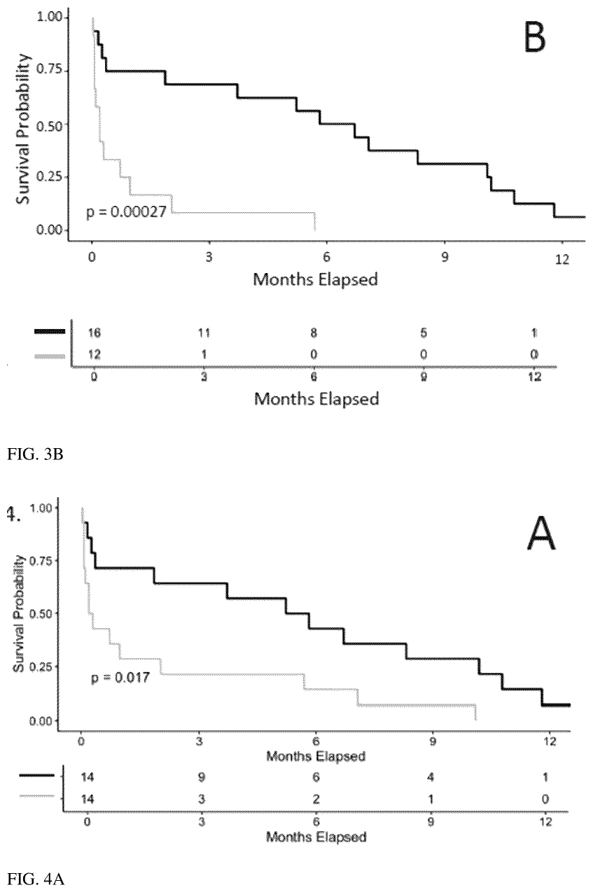

Two KM plots were generated comparing Ugandan CGCI-BLGSP cases with maximum Electrostatic CSs in the upper 50th percentile and the lower 50th percentile Electrostatic CSs based on TCR CDR3-EBV epitope 27992, and 53128 CSs (FIGS. 3A and 3B; Table 2). For complementarity based on EBV epitope 27992, the upper 50th percentile maximum Electrostatic CS Ugandan CGCI-BLGSP cases showed greater OS probability (FIG. 3A; log rank p=0.0021). For complementarity based on EBV epitope 53128, the upper 50th percentile maximum Electrostatic CS Ugandan CGCI-BLGSP cases showed greater OS probability (FIG. 3B; log rank p=0.00027).

### Survival Analysis of Cases Representing Upper and Lower 50th Percentile of CSs Representing the Set of Overlapping T-Cell and B-Cell EBV Epitopes.

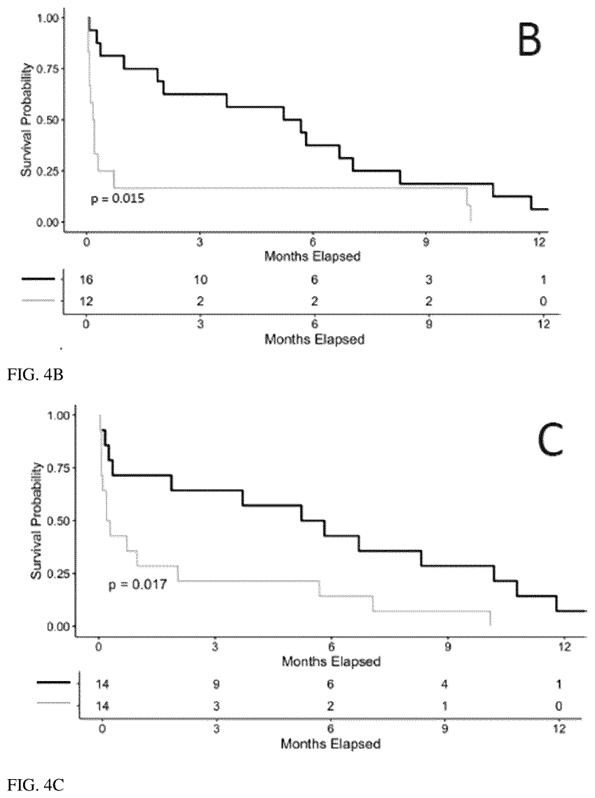

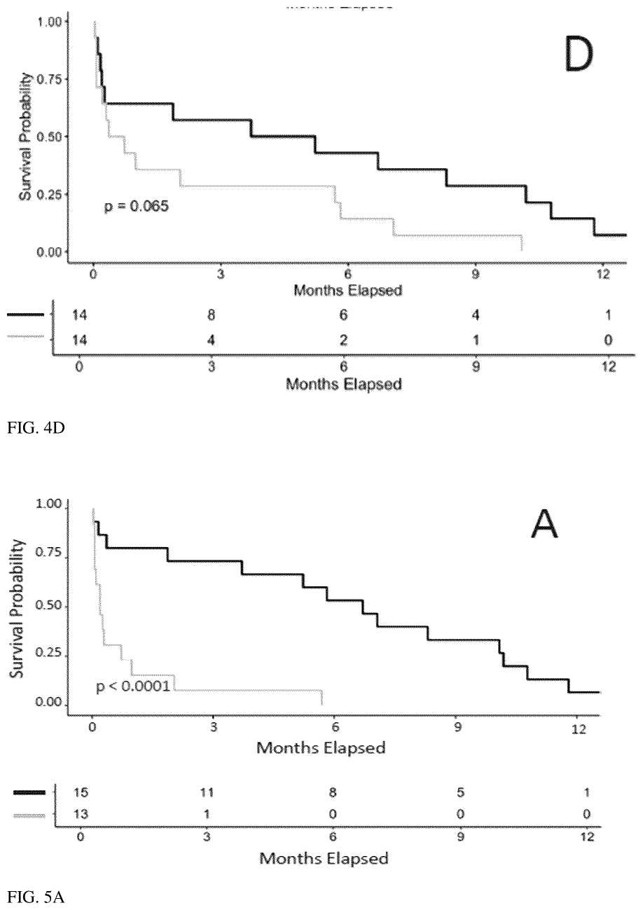

Survival analyses based on CSs represented by the Ugandan CGCI-BLGSP case TCR CDR3s and the 30 EBV epitopes that were indicated as epitopes for both B-cell and T-cell assays in IEDB (Methods) indicated three epitopes that represented unusually high OS probability distinctions based on the Electrostatic CS calculations and one epitope that represented unusually high OS probability distinctions based on the Combo CS calculations. As above, the BL cases were assigned to either the upper or lower 50th percentile group based on the maximum CS for a given case (Tables 3, 4; FIGS. 4A-4D).

TABLE 3
KM analysis output based on Electrostatic CSs of
Ugandan CGCI-BLGSP TRA and TRB CDR3s matched with
T-cell and B-cell assay overlapping EBV epitopes.

| | | | Cases in the | Cases in the |
|---|---|---|---|---|
| | | | upper 50^{th} | lower 50^{th} |
| | Univariate | | percentile of | percentile of |
| | Cox | KM log | Electrostatic | Electrostatic |
| | regression | rank p- | CSs: median | CSs: median |
| epitope | p-value | value | days of survival | days of survival |
| 429189 | 0.009 | 0.0149 | 159 | 5 |
| 194260 | 0.001 | 0.0166 | 177 | 9 |
| 16878 | 0.013 | 0.0166 | 177 | 9 |

Note,
the cases were assigned to the upper or lower 50^{th} percentile based on the maximum Electrostatic CS for the indicated epitope.

TABLE 4
KM analysis output based on Combo CSs of Ugandan
CGCI- BLGSP TRA and TRB CDR3s matched with T-
cell and B-cell assay overlapping EBV epitopes.

| | | | Cases in the | Cases in the |
|---|---|---|---|---|
| | | | upper 50^{th} | lower 50^{th} |
| | Univariate | | percentile of | percentile of |
| | Cox | KM log | Electrostatic | Electrostatic |
| | regression | rank p- | CSs: median | CSs: median |
| epitope | p-value | value | days of survival | days of survival |
| 30951 | 0.041 | 0.0493 | 173 | 9 |

Note,
the cases were assigned to the upper or lower 50^{th} percentile based on the maximum Combo CS for the indicated epitope.

### Survival Analyses of Cases Representing Upper and Lower 50th Percentiles for CGCI-BLGSP TCR CDR3-B-Cell Assay EBV Epitope CS Groups.

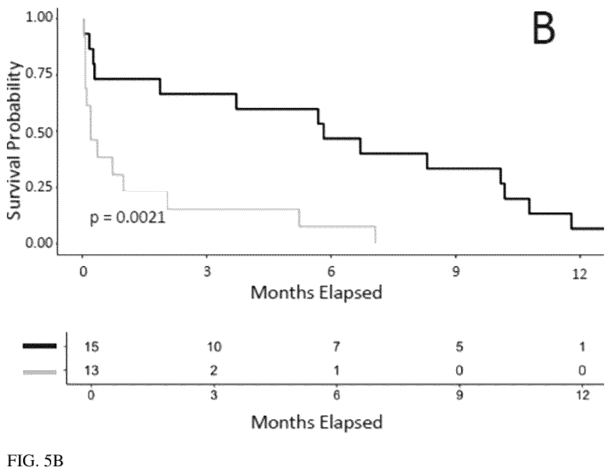

OS probability distinctions of the Ugandan CGCI-BLGSP cases based on Electrostatic CSs for the TCR CDR3s and nine B-cell EBV epitopes (Methods) all represented a log rank p-value of less than 0.03 (Table 5). Specifically, the CGCI-BLGSP cases with a maximum Electrostatic CS in the upper 50th percentile showed increased OS probability in comparison to the CGCI-BLGSP cases with a maximum Electrostatic CS in the lower 50th percentile. The nine EBV epitopes representing these survival probability distinctions had the following IEDB designations: IEDB-95676, IEDB-118800, IEDB-48736, IEDB-114667, IEDB-48738, IEDB-47838, IEDB-55299, IEDB-113211, and IEDB-18587 (Table 5; FIGS. 5A and 5B).

TABLE 5
KM analysis output based on Electrostatic
CSs of Ugandan CGCI-BLGSP TRA and TRB CDR3s
matched with B-cell assay EBV epitopes.

| | | | Cases in the | Cases in the |
|---|---|---|---|---|
| | | | upper 50^{th} | lower 50^{th} |
| | Univariate | | percentile of | percentile of |
| | Cox | KM log | Electrostatic | Electrostatic |
| | regression | rank p- | CSs: median | CSs: median |
| epitope | p-value | value | days of survival | days of survival |
| 95676 | 0.0002 | 9.74E−05 | 204 | 6 |
| 118800 | 0.001 | 0.0291 | 159 | 6 |
| 48736 | 0.001 | 0.0291 | 159 | 6 |
| 114667 | 0.001 | 0.0291 | 159 | 6 |
| 48738 | 0.001 | 0.0291 | 159 | 6 |
| 47838 | 0.002 | 0.0248 | 159 | 6 |
| 55299 | 0.006 | 0.0004 | 177 | 6 |
| 113211 | 0.009 | 0.0149 | 159 | 5 |
| 18587 | 0.019 | 0.0291 | 159 | 6 |

Note,
the cases were assigned to the upper or lower 50^{th} percentile based on the maximum Electrostatic CS for the indicated epitope.

### Discussion

The analyses in this report indicated that those BL cases whose TCR CDR3s showed at least one exact match to an anti-EBV CDR3 showed higher OS probabilities. Furthermore, CS comparisons demonstrated that the means of the maximum Hydro, Electrostatic, and Combo CSs for the CGCI-BLGSP cases with TCR anti-EBV CDR3 matches to the known anti-EBV CDR3s were significantly higher than the means of the corresponding CSs from cases with no anti-EBV TCR CDR3 matches, particularly for CSs based on the epitopes of the EBNA3 protein. Finally, survival analyses also showed a greater OS probability for cases with high TCR CSs based on several T-cell assay epitopes from the IEDB; based on epitopes representing both T-cell and B-cell assays; and based on B-cell assay epitopes. These findings strongly support the notion that BL cases that display a quantifiable, adaptive immune response to EBV epitopes have increased OS probabilities.

### Example 2. Better Outcomes for Ovarian Cancer Associated with the Detection of Anti-EBV CDR3s: Potential Relevance to Diffuse Large B-Cell Lymphoma

Given the ongoing challenges regarding the specific roles of viral infections in cancer etiology or as cancer co-morbidities, this study assessed potential associations between anti-viral T-cell receptor (TCR) complementarity determining region-3 (CDR3s) and clinical outcomes for ovarian cancer. Analyses revealed that patients with exact matches of anti-Epstein-Barr virus (EBV) CDR3 amino acid sequences exhibited better outcomes for both overall and disease-specific survival. However, better outcomes were not observed when assessing anti-viral CDR3s representing cytomegalovirus, influenza A, or Sars-CoV-2. Due to previous occurrences of the occasional misdiagnoses of lymphoma as ovarian cancer, the frequency of anti-EBV CDR3s in lymphoma patients was determined, and these frequencies were relatively high, particularly in diffuse large B-cell lymphoma. These findings underscore the potential value of anti-EBV immune responses in terms of patient outcomes, raise questions about the potential value of anti-EBV immunotherapies, and support further inquiry into the relationship between EBV infection and previously reported cases of ovary-resident lymphoma.

Ovarian cancer ranks as one of the most prevalent gynecological malignancies globally, with significant morbidity and mortality rates. While various risk factors contribute to its etiology, the role of viral infections, particularly Epstein-Barr virus (EBV), remains an area of growing concern, especially in advanced stages of ovarian cancer. Notably, studies have highlighted the geographical variation in ovarian cancer incidence, suggesting potential viral involvement in certain populations.

This study sought to determine the significance of anti-EBV T-cell receptor (TCR) complementarity determining region 3 (CDR3) amino acid (AA) sequences in ovarian cancer patients. As detailed extensively in the results, evaluation of the cancer genome atlas (TCGA) dataset revealed that tumor and blood presence of previously identified, anti-EBV TCR CDR3 AA sequences correlated with better outcomes. The associations of anti-EBV TCR CDR3s with better outcomes for ovarian cancer were further supported by assessing the chemical complementarity of TCR CDR3s to previously identified EBV TCR epitopes, using approaches for assessing adaptive immune receptor, CDR3-cancer epitope, and viral epitope chemical complementarities.

In addition, particularly because of past concerns about detection of primary ovarian non-Hodgkin lymphoma (PONHL), and concerns related to misdiagnosis whereby suspected ovarian epithelial carcinoma was in fact PONHL, the possibility was considered that the anti-EBV CDR3 related results for ovarian cancer were related to PONHL. For example, diffuse large B-cell lymphoma (DLBCL) has been established to be, at least in rare cases, due to malignant transformation by EBV. In de-novo DLBCL cases, a prevalence of EBV-encoded RNA positivity has been reported. Also, Burkitt's lymphoma, particularly in children in sub-Saharan Africa, is exclusively due to malignant transformation by EBV.

### Methods

Recovery of the TRA and TRB Recombination Reads from Genomics File Datasets.

The algorithm applied for the recovery of the TRA and TRB recombination reads from the cancer genome atlas ovarian cancer (TCGA-OV) and stomach adenocarcinoma (TCGA-STAD) exome (WXS) files has been extensively described. The latest version of the software used for mining the TCGA-OV and TCGA-STAD WXS files is freely available at github.com/kcios/vdj_processing. Data representing the full set of TRA and TRB recombination reads is in supporting online material, with the exception of the sequencing reads, which represent controlled access data. The TCGA-OV and TCGA-STAD WXS files were accessed via National Institutes of Health (NIH), database of genotypes and phenotypes (dbGaP) project approval number 6300. The TRA and TRB CDR3s were represented by RNAseq files, in turn representing the TCGA-OV dataset. The TRA and TRB CDR3s derived from the RNAseq files included missing values denoted by symbols ‘*’ or ‘˜’. These incomplete sequences were excluded from the analyses indicated below.

The TCGA-OV RNAseq files were also mined for TRA and TRB recombination reads using the algorithm previously described, i.e., using the same algorithm as was used for mining the TCGA-OV and -STAD WXS files. In addition, the algorithm previously described was used to mine the TRA and TRB recombination reads for the NCICCR-DLBCL and TCGA-DLBC genomics file datasets. The NCICCR-DLBCL dataset was accessed via NIH dbGaP project approval number 22594. The TCGA-DLBC set was obtained and represents only TRA and TRB recombination from TCGA-DLBC RNAseq files. Finally, the algorithm previously described was used to mine the TRA and TRB recombination reads from the WXS and RNAseq files of the CPTAC ovarian cancer dataset. The CPTAC ovarian cancer dataset was accessed via dbGaP.

Assessment of Exact Matches to Anti-Viral CDR3s from the VDJdb.

Anti-viral CDR3 amino acid sequences were retrieved from the VDJdb and compared to the CDR3 amino acid sequences of the translated TRA and TRB recombination reads from the above indicated genomics files using Microsoft Excel functions. In the case of the TCGA-OV analyses in particular, the viruses representing the top four numbers of matches that is, the number of anti-viral TCR CDR3 amino acid sequence matches to the TCR CDR3 amino acid sequences of the TCGA-OV dataset were prioritized for further analyses. Specifically, the highest number of perfect CDR3 amino acid sequence matches to the TCGA-OV TRA and TRB CDR3s were for cytomegalovirus (CMV), totaling 385 matches; Epstein-Barr virus (EBV), with 114 matches; Influenza A (INFA), with 91 matches; and SARS-CoV-2, with 92 matches.

### Chemical Complementarity Scoring of the TRB CDR3-EBV Epitope Pairs.

The Adaptive Match web tool was used to determine the chemical complementarity of the TRB CDR3 amino acid sequences to an EBV epitope obtained from the Immune Epitope Database. This web tool aligned the CDR3 amino acid sequences with the epitope amino acid sequence and calculated chemical complementarity scores (CSs). The TCGA-OV cases representing the upper and lower 50th percentile CSs were determined using a pivot table. For each TCGA-OV case, the maximum CS value was used to sort the cases into the upper and lower 50th percentile cohorts.

### KM Analyses.

For assessment of the TCGA cases, survival analyses were performed with the cbioportal.org web tool. Also, for the analyses of the chemical CSs and the association of the upper and lower 50th percentile groups with survival probabilities, adaptivematch.com was used for confirmation of results. Finally, the results were further confirmed by use of R-studio software. Specifically, the survminer package was used to create and confirm Kaplan-Meier plots and censoring tables for each comparison throughout this report.

### Results

### Initial Assessment of Anti-EBV CDR3s for TCGA-STAD.

To first determine whether there was a detectable outcome difference for cases with anti-EBV TCR CDR3s, based on the exact amino acid sequence match standard, in a cancer setting with a relatively strongly documented EBV component, Kaplan-Meier analyses were used to assess overall survival probabilities for TCGA-STAD cases representing the recovery of TRA and TRB recombination reads from both primary tumor and blood WXS files. The recombination reads were translated, and the TCGA-STAD cases were divided into groups which had (a) TRA or TRB CDR3s (or both) representing an exact amino acid sequence match to previously identified anti-EBV TRA or TRB CDR3s; versus (b) having TRA or TRB CDR3s but no anti-EBV TRA or TRB CDR3s. Results indicated that TCGA-STAD cases represented by the recovery of anti-EBV CDR3s represented a better outcome (Table 6).

TABLE 6
Single time point comparisons of TCGA-STAD cases
with and without anti-EBV CDR3s, based on the
exact AA sequence match standard (Methods).

| | Number of cases | | | |
|---|---|---|---|---|
| | with (upper lines) | | | |
| | and without | | | Two-proportion |
| | (lower | Time | Proportion | comparison |
| Survival | lines) anti-EBV | point | alive/ | p- value |
| parameter | CDR3s | (months) | dead (%) | (Methods) |
| Overall | | | | |
| | 34 | 54.08 | 62.41 | 0.0102 |
| | 293 | 55.56 | 39.43 | |
| Disease | | | | |
| specific | | | | |
| | 32 | 54.08 | 85.01 | 0.0012 |
| | 276 | 55.56 | 55.21 | |
| Progression | | | | |
| free | | | | |
| | 34 | 54.08 | 74.43 | 0.0005 |
| | 295 | 52.21 | 42.89 | |
| Disease free | | | | |
| | 17 | 60.85 | 90.91 | 0.0180 |
| | 179 | 61.22 | 62.13 | |

Note:
All cases represent recovery of at least one TRA or TRB recombination read

### Assessments of Outcome Probabilities for TCGA-OV Cases Representing Exact Amino Acid Sequence Matches to Anti-EBV TCR CDR3s.

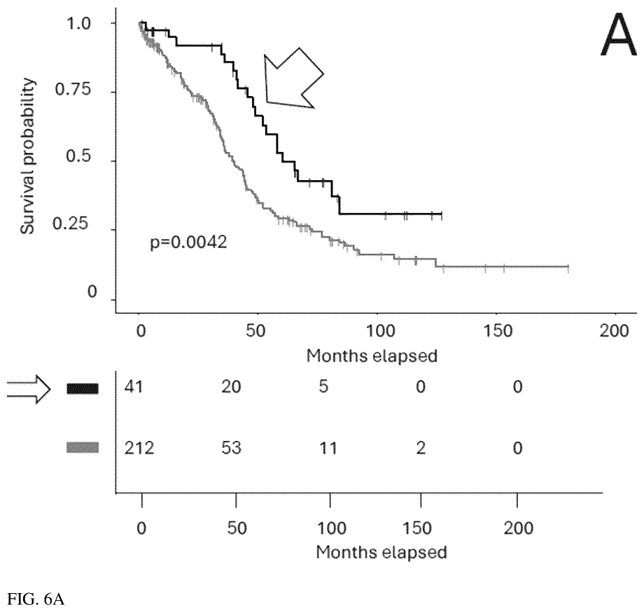

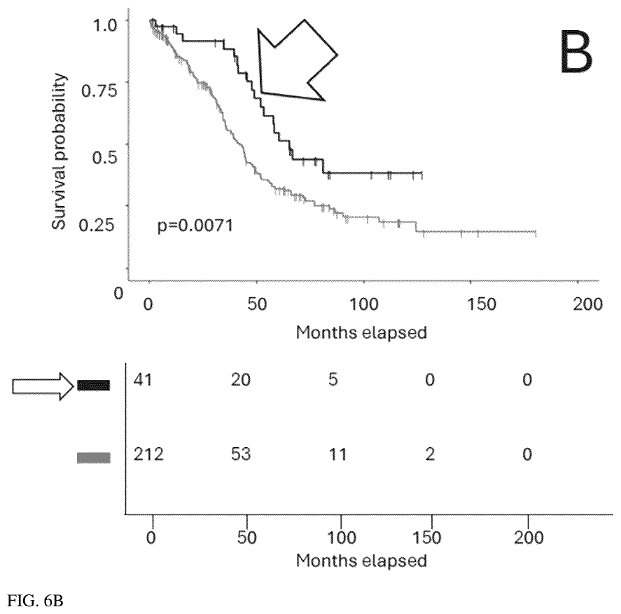

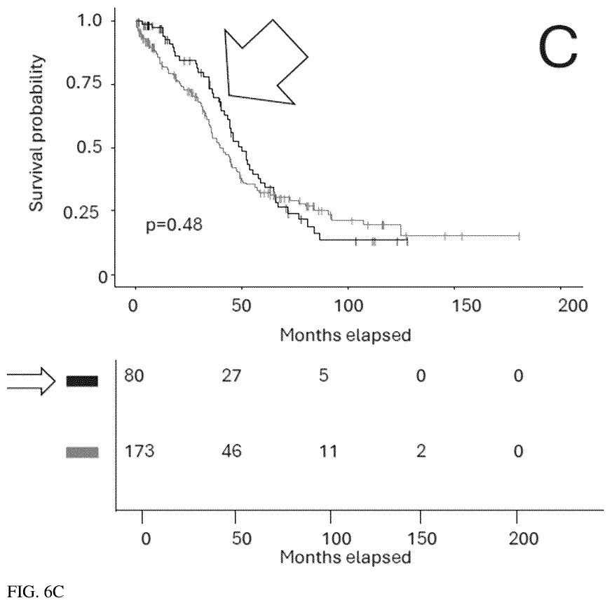

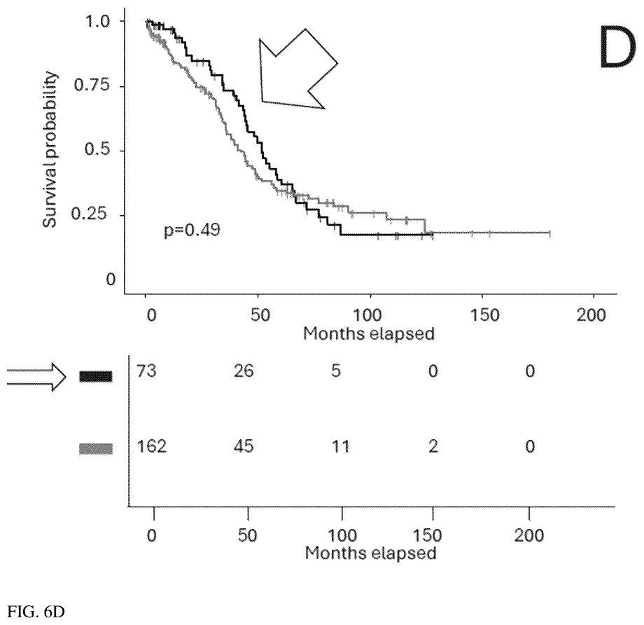

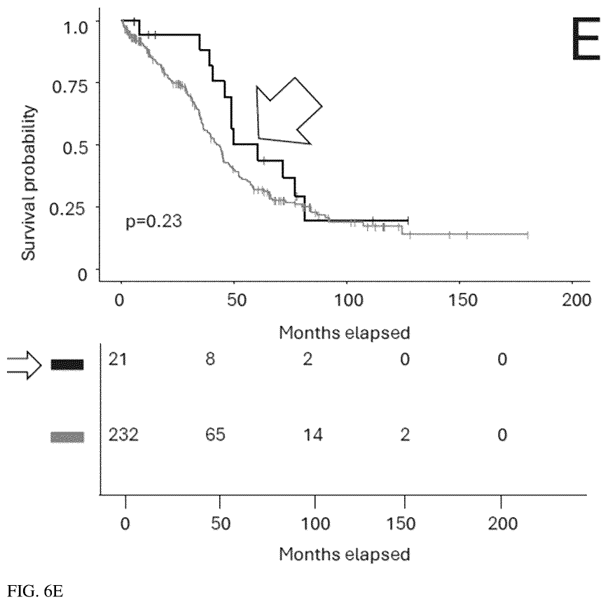

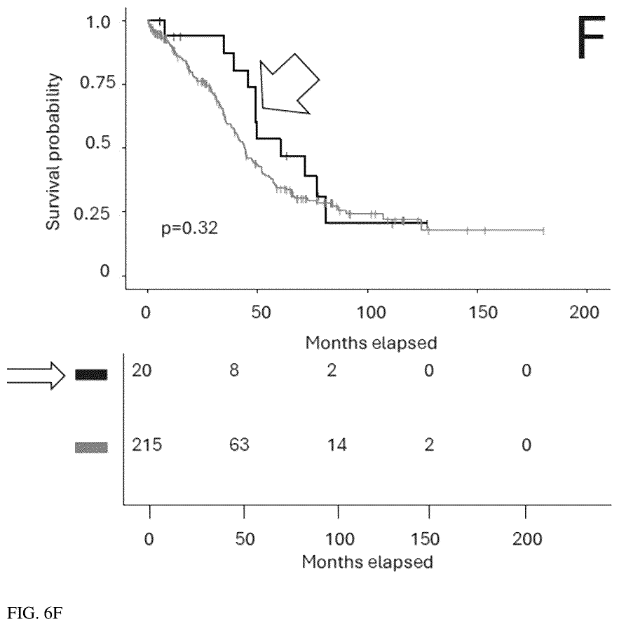

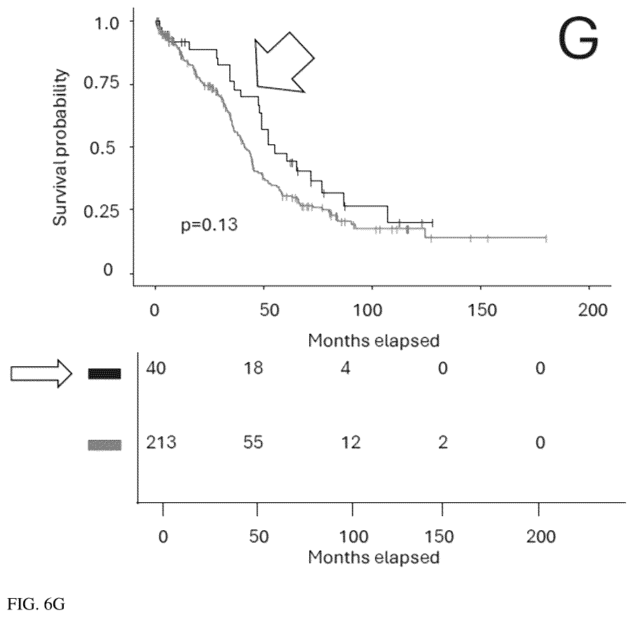

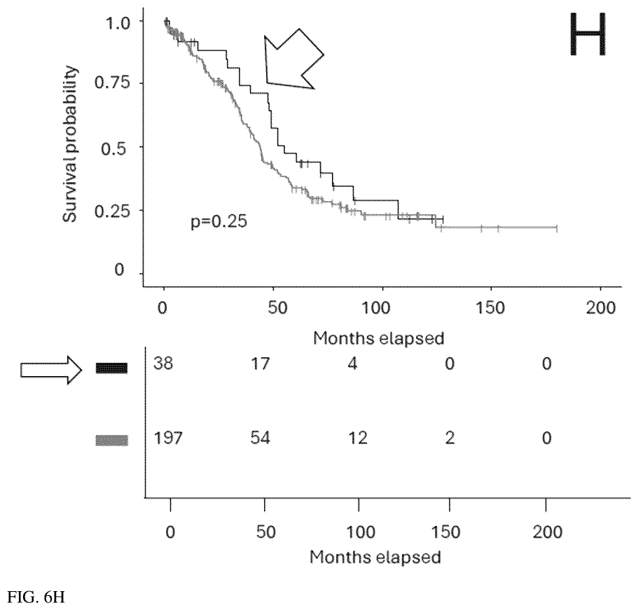

TRA and TRB recombination reads were obtained from WXS files representing all available tissues (blood, tumor, and solid tissue normal, with the latter representing very few samples) (Methods). Cases in which the TRA and TRB CDR3s represented exact amino acid sequence matches to previously identified anti-viral CDR3s were identified. These cases, which carried anti-viral CDR3s, were compared to cases with TRA or TRB recombination reads but lacking anti-viral CDR3s, using Kaplan-Meier analyses. This approach was similar to the one previously described for the analysis of TCGA-STAD cases; however, in this TCGA-OV approach, cases were grouped according to the presence or absence of anti-viral TRA or TRB CDR3s, respectively, for several viruses, including Epstein-Barr virus (EBV) (FIGS. 6A-6H). Results indicated that TCGA-OV cases with either anti-EBV TRA or TRB CDR3s or both types of CDR3s had greater overall survival (OS) and disease-specific survival (DSS) probabilities than cases lacking anti-EBV CDR3s (FIGS. 6A and 6B). In contrast, no statistically significant differences in OS or DSS probabilities were observed when comparing cases with anti-CMV, anti-INFA, or anti-SARS-CoV-2 CDR3s to cases with TRA or TRB recombination reads lacking those anti-viral CDR3s (FIGS. 6C-6H).

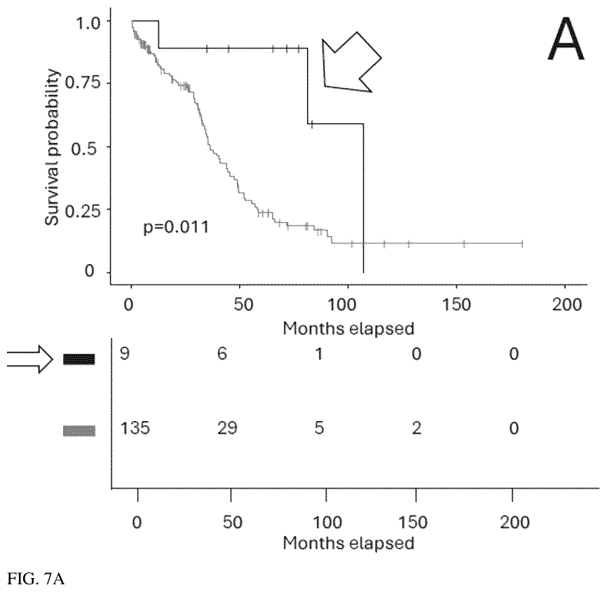

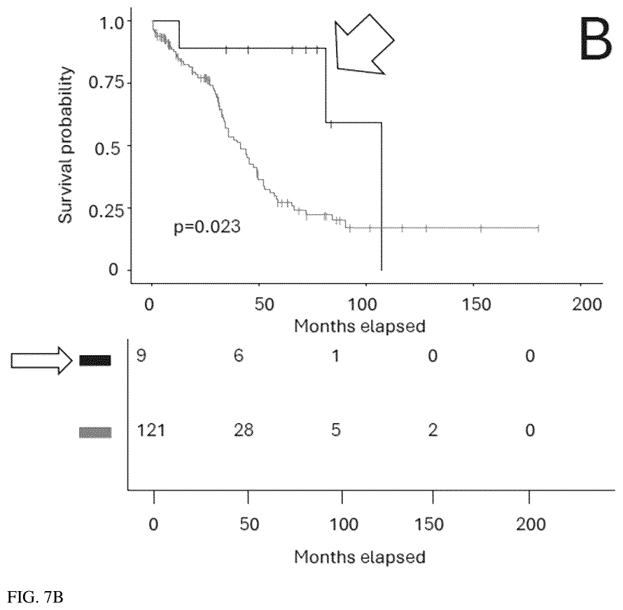

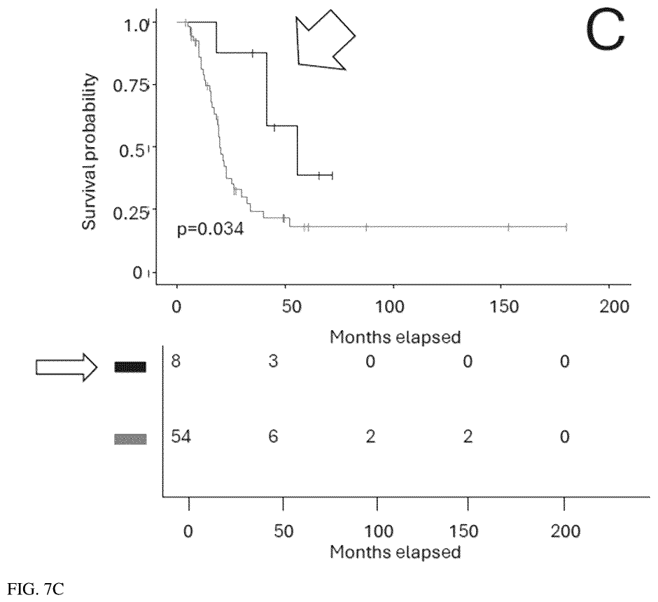

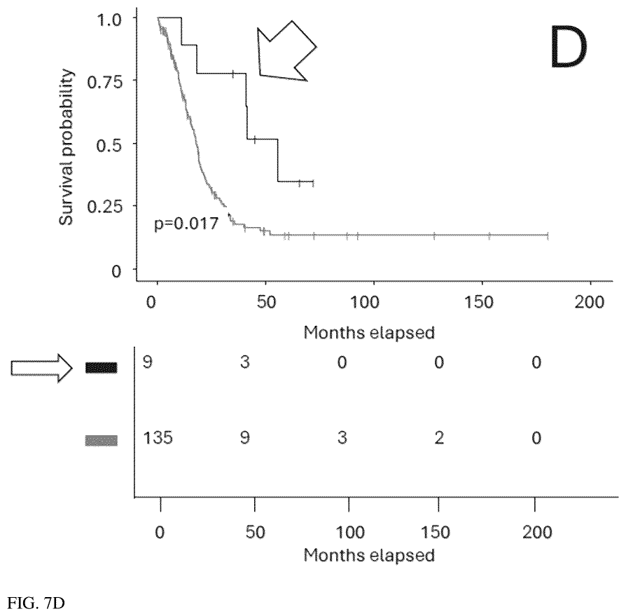

Next, outcome distinctions were assessed based solely on anti-EBV TRA and TRB CDR3s recovered from tumor WXS files, rather than distinguishing cases based on combined recoveries of CDR3s from both tumor and blood WXS files, as in the preceding analysis. Results indicated that TCGA-OV cases with tumor-resident anti-EBV CDR3s had higher OS, DSS, disease-free survival (DFS), and progression-free survival (PFS) probabilities compared to cases lacking tumor-resident TRA or TRB anti-EBV CDR3s, based on exact amino acid sequence matching (FIGS. 7A-7D).

### Assessment of Outcome Probabilities for TCGA-OV Cases Representing Exact Amino Acid Sequence Matches for Anti-EBV TCR CDR3s Using Tumor RNAseq Files.

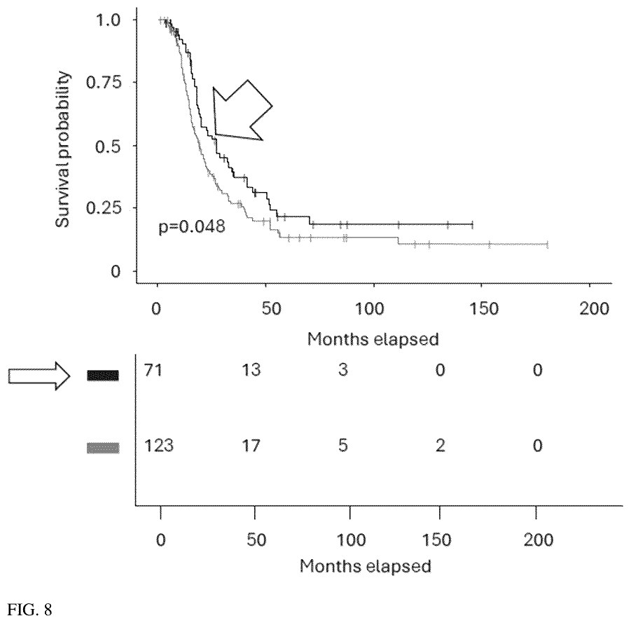

TCGA-OV cases with TRA or TRB recombination reads from tumor RNAseq files, where the recombination reads represented exact amino acid sequence matches to previously identified anti-EBV CDR3s, were identified. These cases were compared to those with TRA or TRB recombination reads but lacking anti-EBV CDR3s. Results indicated that TCGA-OV cases with anti-EBV TRA or TRB CDR3s had greater disease-specific survival (DSS) probabilities than those lacking such CDR3s (FIG. 8A). It is noted that the RNAseq-derived TRA and TRB CDR3s were initially obtained by an independent group using a distinct algorithm for recovering recombination reads. In addition, TRA and TRB recombination reads were extracted from the TCGA-OV RNAseq files using the algorithm described in the Methods. Cases representing anti-EBV TCR CDR3s were identified, and a Kaplan-Meier analysis showed a better outcome for these cases based on a single significant time point difference (p=0.03 at the 40-month mark), compared to cases with TRA or TRB recombination reads but no anti-EBV TCR CDR3 matches (FIG. 8B).

### Assessment of Outcome Probabilities of TCGA-OV Cases Representing the Upper or Lower 50th Percentile Groups for TRB CDR3-ICP27 Epitope*193996 Combo Complementarity Scores (CSs).

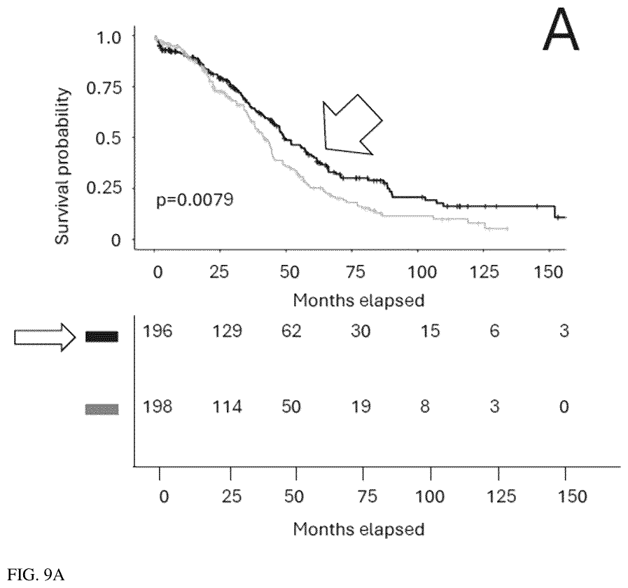

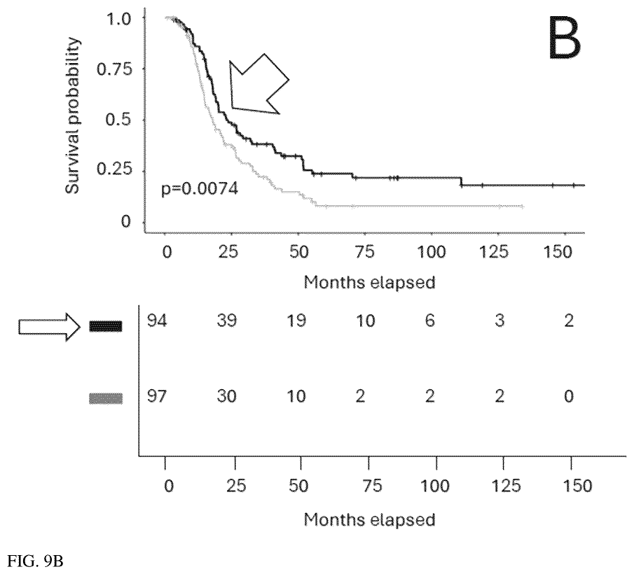

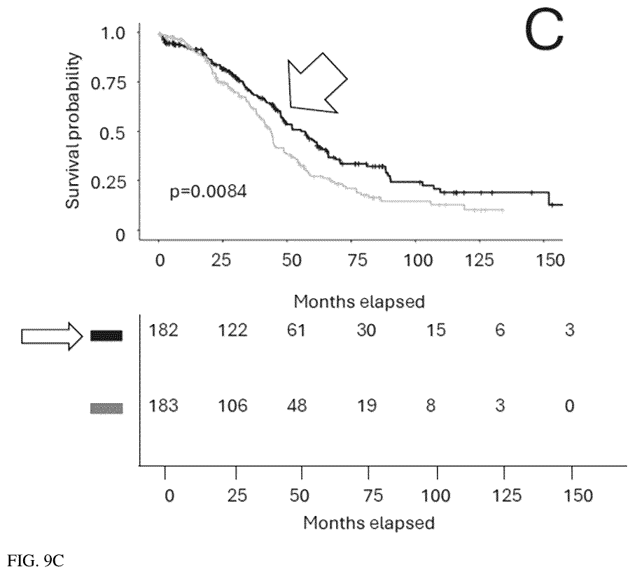

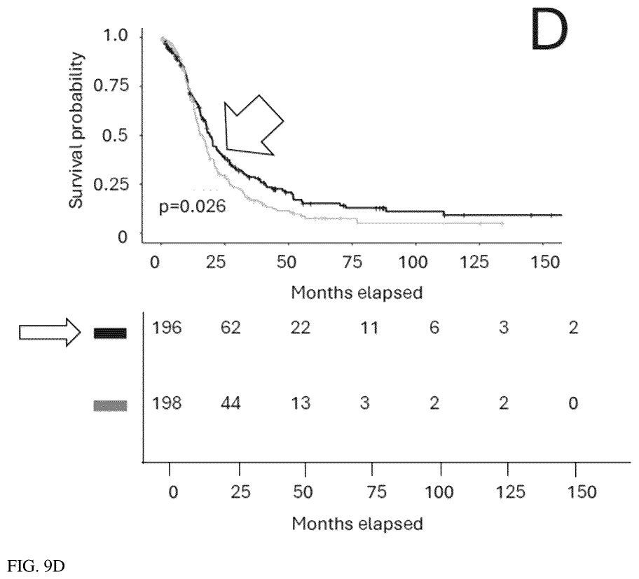

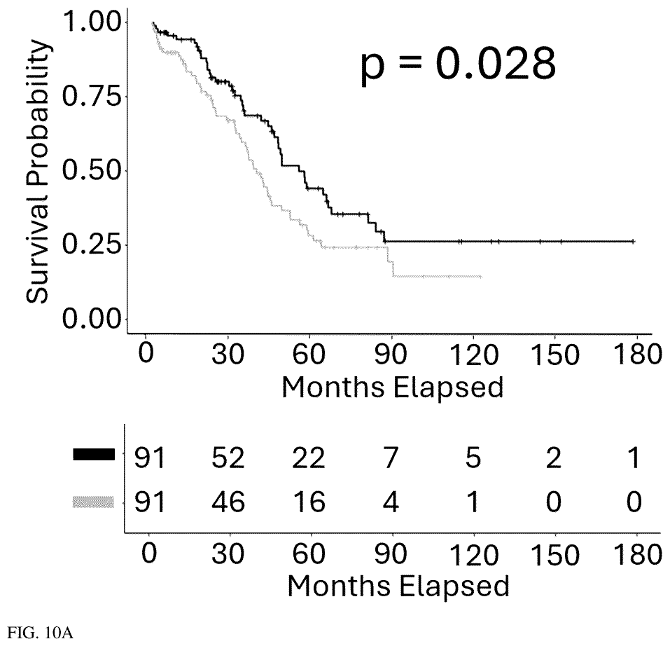

As an alternative approach to assessing the association of TCR anti-EBV CDR3s with survival distinctions, chemical complementarity scores (CSs) were obtained for the TCGA-OV TRB CDR3s and an EBV epitope designated as ICP27 Epitope193996 in the Immune Epitope Database (IEDB) (Methods). TCGA-OV cases representing the upper and lower 50th percentile groups for the Combo CSs of TRB CDR3-ICP27 Epitope were identified using the adaptivematch.com web tool (Methods). Results indicated that the TCGA-OV cases in the upper 50th percentile of Combo CSs had greater disease-free survival (DFS), overall survival (OS), disease-specific survival (DSS), and progression-free survival (PFS) than the cases in the lower 50th percentile (FIGS. 9A-9D).

### Assessment of Anti-EBV TCR CDR3s Among DLBCL Cases.

In light of the unresolved issue of misdiagnosing primary ovarian non-Hodgkin lymphoma (PONHL) as ovarian carcinoma, addressed in the Introduction and further in the Discussion, the frequency of anti-EBV TCR CDR3s was assessed among cases of diffuse large B-cell lymphoma (DLBCL), a lymphoma type known to be mistaken for ovarian carcinoma. Using the NCICCR-DLBCL dataset (Methods), TRA and TRB recombination reads were analyzed to identify exact amino acid sequence matches to previously reported anti-EBV CDR3s. Among cases with recovered TRA recombination reads from NCICCR tumor RNAseq files, 252 out of 455 (approximately 55%) had anti-EBV CDR3 matches. For TRB recombination reads from RNAseq, 49% of the cases showed anti-EBV matches (Table 7). In tumor WXS files from the same dataset, 56% of the NCICCR-DLBCL cases with recovered TRA recombination reads had anta-EBV CDR3s, while 19% of those with TRB recombination reads showed anti-EBV matches (Table 7). In the second dataset, TCGA-DLBC (Methods), 26 out of 47 cases with TRA recombination read recoveries from tumor RNAseq files (approximately 55%) had anti-EBV CDR3s. For TRB recombination reads in the same dataset, 31% of cases had anti-EBV matches (Table 7).

TABLE 7
Anti-EBV CDR3, exact AA sequence matches to TCR CDR3s recovered
from cases of diffuse large B-cell lymphoma (DLBCL).

| | |
|---|---|
| Case count for | |
| tumor RNAseq- | |
| derived adaptive IR | Case count for tumor |
| recombination | WXS- derived adaptive |
| reads | IR recombination reads |

NCICCR-DLBCL

| | | |
|---|---|---|
| TRA | 252/455 | 238/426 |
| TRB | 221/455 | 74/400 |

TCGA-DLBC

| | | |
|---|---|---|
| | Tumor RNAseq | Tumor WXS, not |
| TRA | 20/48 | applicable |
| TRB | 15/48 | |
| | Tumor RNAseq | |
| TRA | 26/47 | |
| TRB | 15/48 | |

Note,
cases in the denominator represent all cases with an adaptive immune receptor (IR) recombination read recovery. NA, not applicable: adaptive IR recoveries from the TCGA diffuse large B-cell lymphoma WXS file set were too few for analyses.

### Detection of Anti-EBV CDR3s in a Second Ovarian Cancer Dataset.

The CPTAC-OV dataset, representing both RNAseq and WXS files with TCR recombination reads (Methods), was analyzed to determine the frequency of cases with exact amino acid sequence matches to anti-EBV CDR3s in a second ovarian cancer dataset. Among the CPTAC-OV WXS files, 32 out of 130 cases showed recovery of TCR recombination reads with anti-EBV matches, representing approximately 25% of the CPTAC-OV cases (Table 8). Additionally, 47 out of 133 cases from CPTAC-OV RNAseq files showed anti-EBV CDR3 matches, representing approximately 35% of the cases (Table 8).

TABLE 8
Anti-EBV TCR CDR3s in the CPTAC ovarian cancer dataset.

| Anti-EBV CDR3s, based | Anti-EBV CDR3s, based |
|---|---|
| on exact AA sequence | on exact AA sequence |
| matches, for TCR | matches, for TCR |
| recombination reads | recombination reads |
| obtained from WXS files | obtained from RNAseq files |
| 32/130 | 47/133 |

Note,
cases in the denominator represent all cases with an adaptive immune receptor (IR) recombination read recovery.

### Discussion

This study provides new insights into a potential role for anti-EBV TCR CDR3s in ovarian and stomach cancer. Patients with ovarian cancer (TCGA-OV) who exhibited anti-EBV TCR CDR3s were found to have significantly better overall survival (OS), disease-specific survival (DSS), disease-free survival (DFS), and progression-free survival (PFS) than those without detectable anti-EBV CDR3s. These findings align with existing research implicating EBV in cancer pathogenesis and emphasize the relevance of TCR profiling for predicting patient outcomes.

One important issue to consider is the potential misdiagnosis of lymphoma as ovarian epithelial carcinoma, specifically in cases involving primary ovarian non-Hodgkin lymphoma (PONHL). Such misdiagnoses could partly account for the improved survival rates seen in patients with anti-EBV TCR CDR3s, as lymphomas generally have a more favorable prognosis than ovarian epithelial carcinoma and are often associated with EBV. This consideration may also be relevant to stomach cancer and possibly to other cancer types.

While the data do not allow a clear distinction between an anti-EBV immune response against ovarian epithelial carcinoma and a misdiagnosed PONHL, other findings from this study point to a higher-than-expected presence of anti-EBV TCR CDR3s in diffuse large B-cell lymphoma (DLBCL) one of the most common PONHL subtypes mistakenly diagnosed as ovarian epithelial carcinoma (Table 7). The elevated proportion of anti-EBV CDR3s in DLBCL suggests that EBV may play a more prominent role in DLBCL than previously assumed.

These observations underscore the importance of further research into the relationship between EBV and DLBCL and also into the biological mechanisms underlying improved outcomes in ovarian cancer cases where anti-EBV CDR3s are present. Deeper understanding of this connection may inform the development of more effective, EBV targeted therapies for DLBCL and perhaps for a subset of ovarian cancer cases as well.

### Example 3. Higher Chemical Complementarity of EBV Epitopes and IGH CDR3s Correlates with Better Outcomes for Lymphoma and Ovarian Cancer

The current invention also describes the relationship between Epstein-Barr virus (EBV) epitopes and tumor-resident immunoglobulin heavy locus (IGH) CDR3 amino acid (AA) sequences sourced from cancers with a link to EBV. In the case of ovarian cancer, the results revealed that higher chemical complementarity between certain EBV epitopes and IGH CDR3 AA sequences was linked to better outcomes. Additionally, IGH CDR3 and EBV epitope chemical complementarity was examined for Burkitt lymphoma (BL) and Diffuse large B-cell lymphoma (DLBCL). For both BL and DLBCL, higher chemical complementarity to certain EBV epitopes was associated with improved overall survival probabilities.

Ovarian cancer is the deadliest form of gynecologic cancer, with high recurrence and low survival rates, especially for late-stage disease. Previous studies have suggested a role for viral infections, such as Epstein-Barr virus (EBV), and the development of ovarian cancer, but this has yet to be examined in an extensive manner. The current invention assesses the importance of chemical complementarity between EBV epitopes and immunoglobulin heavy chain (IGH) complementarity-determining region 3 (CDR3) amino acid (AA) sequences obtained from ovarian cancer patients. It is also known that the development of certain lymphomas can be associated with an EBV infection, such as Diffuse large B-cell lymphoma (DLBCL). Additionally, childhood Burkitt lymphoma (BL) is almost always associated with EBV in sub-Saharan Africa. Thus, the current invention evaluated the chemical complementarity of IGH CDR3s and EBV epitopes for these lymphoma settings. Overall, results showed that higher chemical complementarity between EBV epitopes and IGH CDR3s indicated better outcomes for the ovarian cancer and lymphoma datasets.

### Methods

Mining of the IGH Recombination Reads from Tumor-Resident RNAseq Files.

The algorithm and script for recovery of the IGH recombination reads have been extensively described and benchmarked. The latest iteration of the computer code is freely available at github.com/kcios/vdj_processing. This processing algorithm was applied to RNAseq files for the following datasets: The Cancer Genome Atlas-Ovarian Serous Cystadenocarcinoma (TCGA-OV); Clinical Proteomic Tumor Analysis Consortium-Ovarian Serous Cystadenocarcinoma (CPTAC-OV); Cancer Genome Characterization Initiative: Burkitt Lymphoma Genome Sequencing Project (CGCI-BLGSP); National Cancer Institute's Center for Cancer Research-Diffuse large B-cell lymphoma (DLBCL). These datasets were accessed according to NIH database of Genotypes and Phenotypes (dbGaP) project approval numbers as follows: TCGA-OV, 6300; CPTAC-OV, 31752; CGCI-BLGSP, 31203; NCICCR-DLBCL, 22594. In addition, IGH recombination reads for TCGA-OV, as a second TCGA-OV IGH recombination read dataset. This latter dataset resulted from a distinct algorithm (from a separate research group) used in the recovery of the IGH recombination reads, and in this report, the dataset will be referred to as the IGH CDR3 dataset from the Thorsson algorithm.

### Chemical Complementarity Scoring for the IGH CDR3s and EBV Epitopes.

For each dataset, IGH CDR3 AA sequences associated with case IDs were sorted by the frequency of repeats using the Excel COUNTIF function. The top approximately 2000 most frequently repeated CDR3s were then selected for further analysis, using the Adaptive Match web tool. The “2000” number is approximate because CDR3s were included based on higher versus lower frequency of occurrence. However, when the number 2000 was reached within a specific frequency value, the collection of CDR3s continued until the frequency value changed. Next, the Immune Epitope Database (IEDB) was used to identify all EBV epitopes from linear peptides and a B-cell assay. The top 100 EBV epitopes were identified based on the highest number of literature references. Overall survival data were obtained from clinical files available on public websites. Specifically, survival data for the TCGA-OV dataset were accessed through cbioportal.org, while data for the NCICCR-DLBCL and CGCI-BLGSP datasets were retrieved from the Genomic Data Commons (GDC) data portal. Of note, the analyses of the CGCI-BLGSP dataset included Ugandan patients only for this report. Then, the IGH CDR3s, EBV epitopes, and overall survival data were inputted into the Adaptive Match web tool. Using the Adaptive Match web tool, the CDR3 amino acid sequences were matched with the EBV epitope amino acid sequences via a “sliding window” to determine the respective chemical complementarity scores (CSs). The Adaptive Match web tool outputs the list of epitopes representing the statistically significant survival distinctions for the upper and lower 50th percentile case groups based on the maximum IGH CDR3-epitope CS for each case. The Adaptive Match web tool outputs Hydro, Electrostatic, and Combo CSs, but only the Hydro CSs were used in this report. The epitopes representing the statistically significant survival distinctions were indicated by both a univariate Cox-regression p-value and a log rank p-value representing a Kaplan-Meier (KM) analysis. An Excel pivot table representing all the raw CSs for any given IGH CDR3 epitope was used to identify the upper and lower 50th percentile case groups, based on the maximum CS for each case. This process allowed verification of the Adaptive Match survival distinction outputs, with subsequent use of the cbioportal.org web tool or the GDC data portal for the KM analyses, as described in further detail below. Also, R-studio was used for further verification and preparation of all KM analysis figures in this report.

Statistical Summary for the Three Datasets where IGH CDR3-EBV Epitope CSs Represented Survival Distinctions.

For the assessment of the TCGA-OV IGH CDR3-CSs, two independent IGH CDR3 datasets were consistent for the same results. Specifically, one CDR3 dataset emphasized the identity of the V and J in the RNAseq recombination reads, with the CDR3 then established as either productive or unproductive. The second algorithm emphasized the identification of CDR3s followed by a low stringency acceptance of candidate V and J sequences. Thus, the TCGA-OV dataset results were verified by an independent dataset representing a distinct approach to identifying the CDR3s by another research group, which meets a replicative set statistical standard.

As for the assessment of the CGCI-BLGSP IGH CDR3-EBV epitopes, a size-matched control set of epitopes representing other viruses except for EBV, and representing the same number of inputs as the experimental EBV set, did not yield any Hydro CS KM log rank p-values below 0.05, with the lowest KM log rank p-value as 0.346. When EBV epitopes were used, the lowest KM log rank p-value was 0.075 for the EBV epitope IEDB86944. In other words, in neither case non-EBV epitopes or EBV epitopes was the standard for statistical significance achieved; however, it was evident that a survival trend was detected in the case of the EBV epitope IEDB86944, but not in the case of the control epitope set.

As for the assessment of the NCICCR-DLBCL IGH CDR3-EBV epitopes, the CGCI-BLGSP approach was repeated. However, for this NCICCR-DLBCL dataset, there were many more cases and CDR3s. Thus, the statistical significance of the resulting Hydro CS-related distinctions using the EBV epitopes contrasted with the implied distinctions using the control, non-EBV epitopes (to account for multiple testing represented by the EBV epitope set). Analysis with non-EBV epitopes did not yield any Hydro CS KM log rank p-values below 0.05. The non-EBV epitopes were arbitrarily labeled 1-100, and two of the epitopes with the lowest Cox-regression p-values and two of the epitopes with the lowest KM log rank p-values are indicated in Table 9. This contrasts with the Hydro CS-related survival distinctions observed with the EBV epitopes, where several EBV epitopes yielded Hydro CS KM log rank p-values below 0.05. This contrast indicates that the EBV epitopes represented a conventional standard of statistical significance independent of multiple testing, and as such, these EBV epitopes were termed for this report “high confidence” epitopes when assessing overall survival probabilities.

### Kaplan-Meier (KM) Analyses.

As noted above, the case IDs associated with the upper and lower 50th percentile groups for chemical complementarity scores (CSs) were inputted into cbioportal.org or the GDC data portal. KM curves for TCGA-OV cases were generated using the web tool at cbioportal.org, while KM curves for the CGCI-BLGSP and the NCICCR-DLBCL cases were generated using the web tool at the GDC data portal. (Note: the survival time parameter for the NCICCR-DLBCL dataset was only available as “days-to-last-follow-up.”)

### Gender Stratification of the NCICCR-DLBCL Dataset.

The gender distribution was analyzed for the upper and lower 50th percentile case groups based on Hydro CSs for NCICCR-DLBCL IGH CDR3s and multiple EBV epitopes from the IEDB. The number of female and male cases in each group was recorded. The average percentage of male cases in the upper 50th percentile group was compared to the average percentage of male cases in the lower 50th percentile group (n=268). MedCalc.org was used to perform a two-proportion test to compare these percentages. This analysis was also repeated for the average percentage of female cases in the upper and lower 50th percentile groups (n=168). Results are reported in Table 10.

### Tumor Staging and Chi-Squared Tests.

After dividing the TCGA-OV cases into upper and lower 50th percentile groups based on Hydro CSs, case IDs were matched to their tumor stages from the clinical file. A linkage of tumor grade to the case IDs was applied to the upper and lower Hydro CS groups for the TCGA-OV dataset, with the Hydro CSs based on the IGH CDR3s. The linkage of tumor stages to the case IDs representing the upper and lower 50th percentile Hydro CS groups was also performed for the NCICCR-DLBCL dataset and CPTAC-OV dataset. The epitopes used to define the upper and lower 50th percentile CS groups are indicated in Results. A Chi-squared statistical analysis method was used to determine whether there was a statistically significant association between either tumor staging (for CPTAC-OV and NCICCR-DLBCL) or tumor grade (for TCGA-OV) and a case group (upper versus lower 50th percentile groups). The Chi-squared tests were performed for the three datasets described above, and results for the TCGA-OV and NCICCR-DLBCL sets are indicated in Table 11. In all tests, the null hypothesis stated that there was no difference in cancer staging (or grade) between the upper versus lower case groups, while the alternative hypothesis stated that there was a difference. Expected frequencies were calculated using Excel and compared to observed frequencies of tumor staging in each case group. A Chi-squared value was obtained and compared to the critical value to decide whether to reject the null hypothesis. This Chi-squared value was then converted into a p-value.

### Results

### Assessment of Survival Probabilities of TCGA-OV Cases Representing the Upper and Lower 50th Percentile Case Groups for Hydro CSs Based on IGH CDR3s and IEDB*30951.

TCGA-OV IGH recombination sequencing reads representing IGH CDR3s were mined from RNAseq files. The Adaptive Match web tool was used to obtain Hydro CSs for the TCGA-OV IGH CDR3s and the top 100 EBV B-cell epitopes based on the highest number of literature references for each epitope as indicated by the IEDB. Upper and lower 50th percentile case groups were then established (for each epitope) based on the maximum Hydro CS for each TCGA-OV case. The Adaptive Match web tool outputted survival probability distinctions with a univariate Cox regression p-value representing the hazard, or anti-hazard, of the increasing Hydro CS values, and a log rank p-value representing a KM analysis.

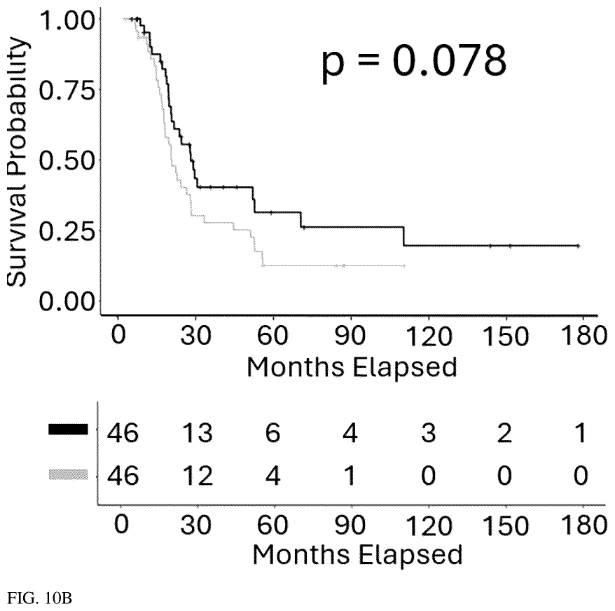

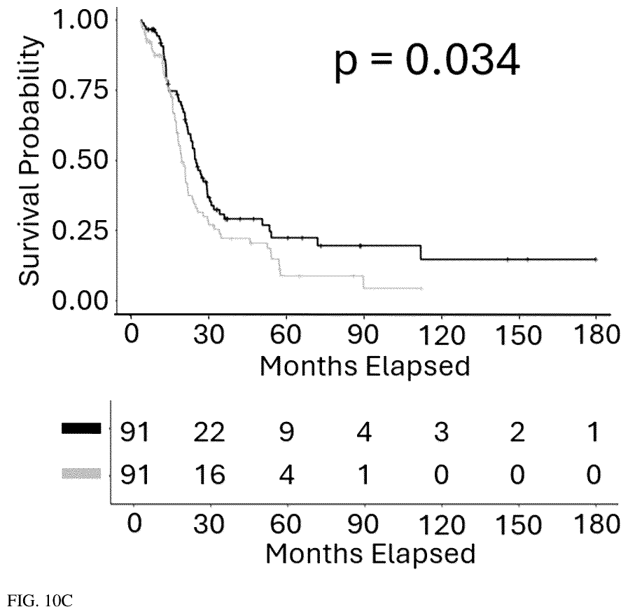

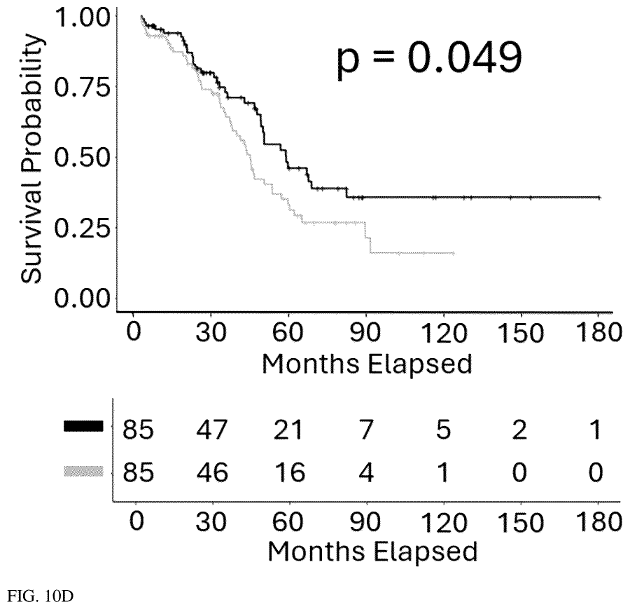

The most significant survival distinction, based on the Cox p-value and the reduced hazard of increasing Hydro CSs, was represented by the EBV epitope IEDB30951. Furthermore, TCGA-OV cases representing the upper and lower 50th percentile case groups for the Hydro CSs represented by IEDB30951 also showed distinct overall survival (OS), disease-free survival (DFS), progression-free survival (PFS), and disease-specific survival (DSS) probabilities in the KM analyses, with the upper 50th percentile case group having a higher survival probability for all the indicated survival parameters (FIGS. 10A-10D).

Note, in the case of DFS, there was a trend rather than a standard result for statistical significance. Also note, the KM analyses of the figures represent an independent assessment and confirmation of results using R-studio following the preliminary findings with the use of the Adaptive Match web tool.

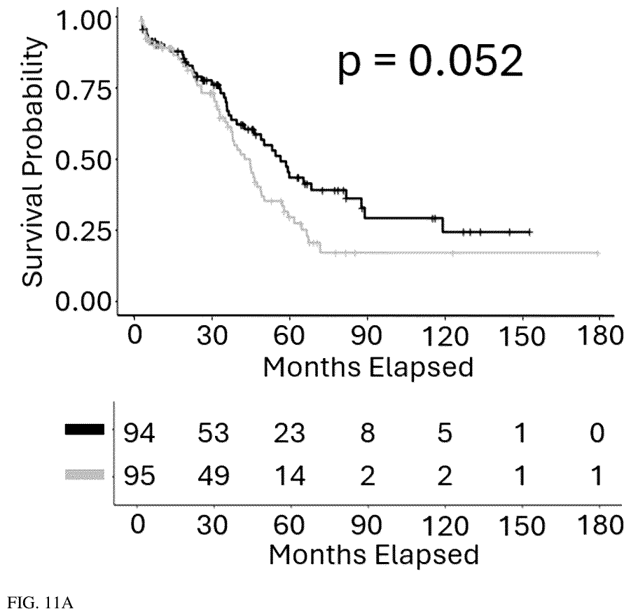

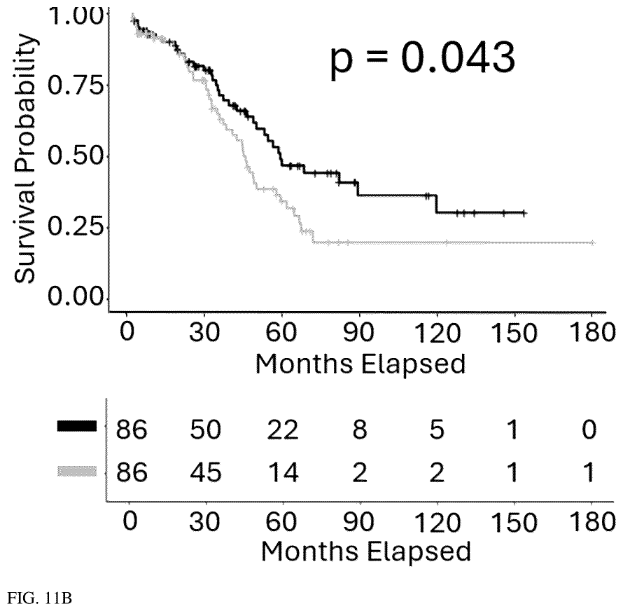

Next, the assessment was repeated using TCGA-OV IGH CDR3s from the mining of recombination reads from RNAseq files by an independent research group using the distinct Thorsson algorithm. Results indicated that the Hydro CSs based on those IGH CDR3s and the IEDB30951 epitope were again associated with higher survival probabilities. Specifically, results indicated that TCGA-OV cases representing the upper 50th percentile of Hydro CSs for IEDB30951 showed greater OS and DSS probabilities than did the cases representing the lower 50th percentile (FIGS. 11A and 11B). The OS results were trending, while DSS results were statistically significant. In sum, a replicative, independent IGH CDR3 dataset was consistent with the association between high Hydro CSs, based on the IEDB*30951 epitope, and better survival probabilities.

### Assessment of Survival Probabilities of CGCI-BLGSP Cases Representing the Upper or Lower 50th Percentile Case Groups for Hydro CSs Based on IGH CDR3s and the IEDB*86944 Epitope.

Keeping in mind that sub-Saharan African BL is almost always exclusively due to EBV, an assessment was conducted to determine whether the CS approach with IGH CDR3s and EBV B-cell epitopes would reveal survival probability distinctions for a set of Ugandan BL cases. Hydro CSs for the CGCI-BLGSP IGH CDR3s and the top 100 IEDB EBV epitopes were obtained using the Adaptive Match web tool. This analysis was limited to those cases with available data for the “days-to-death” parameter.

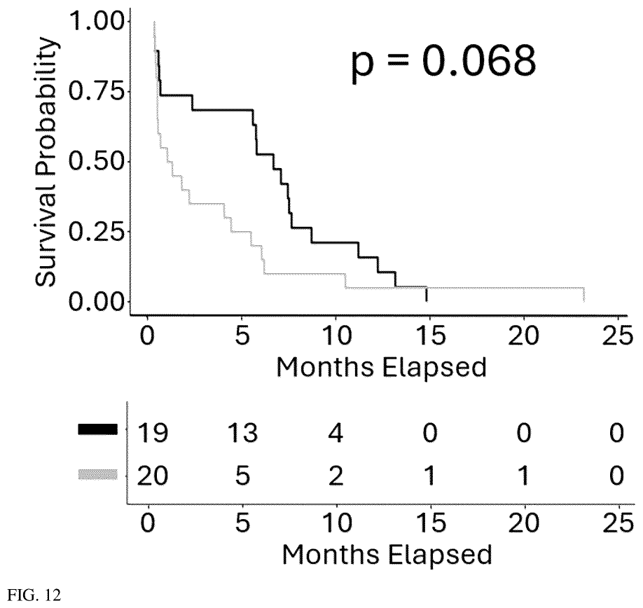

When using this survival time parameter, the Hydro CSs based on the EBV epitope IEDB86944 represented a trend toward survival distinctions. Specifically, results showed that CGCI-BLGSP cases representing the upper 50th percentile of Hydro CSs obtained with the IGH CDR3s and the IEDB86944 epitope had greater OS probabilities than did the cases representing the lower 50th percentile of Hydro CSs (FIG. 12). However, no such trend was indicated with Hydro CSs based on the CGCI-BLGSP IGH CDR3s and any of the control, non-EBV epitopes.

### Assessment of Survival Probabilities of NCICCR-DLBCL Cases Representing the Upper or Lower 50th Percentile Case Groups for Hydro CSs Based on IGH CDR3s and Multiple IEDB EBV Epitopes.

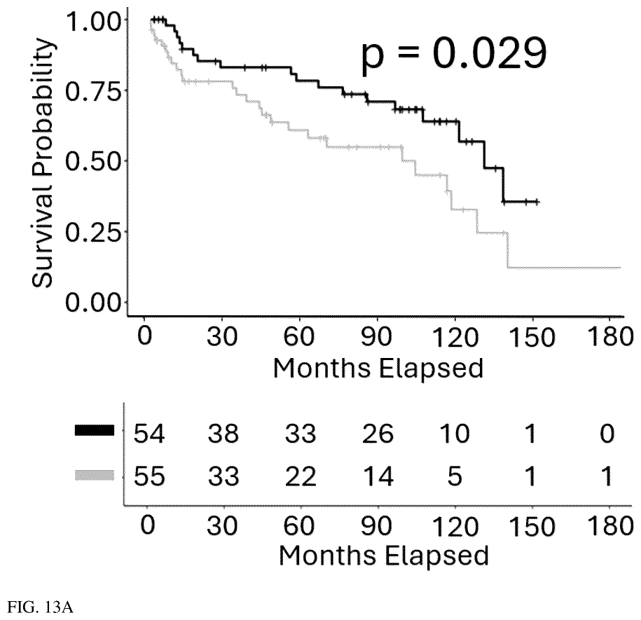

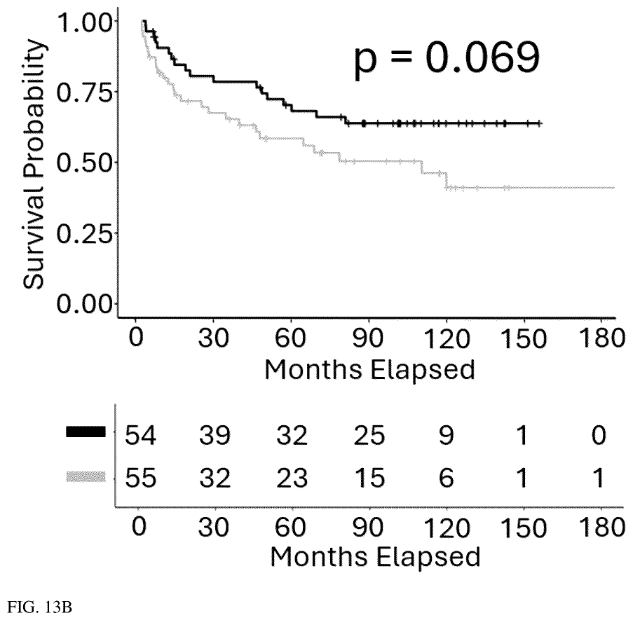

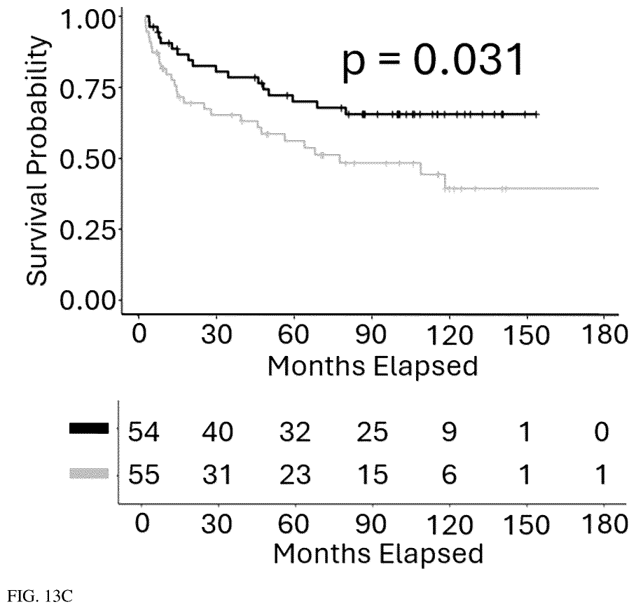

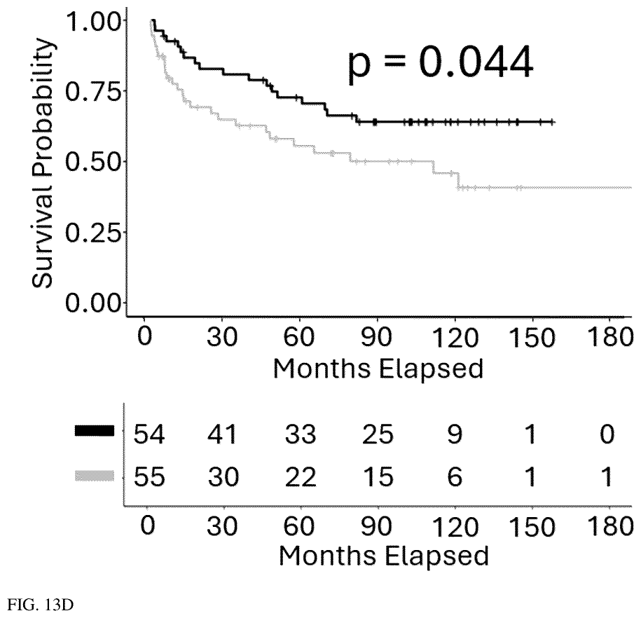

IGH recombination sequencing reads representing the NCICCR-DLBCL CDR3s were mined from RNAseq files using the algorithm described in the Methods section. Hydro CSs for the NCICCR-DLBCL IGH CDR3s and the top 100 IEDB EBV epitopes were obtained using the Adaptive Match web tool. The EBV epitope IEDB144799 was shown to be a high-confidence epitope for survival distinctions (Table 9, including Cox univariate p-values). Results indicated that cases representing the upper 50th percentile of Hydro CSs calculated with the IGH CDR3s and the EBV B-cell epitope IEDB144799 had greater overall survival (OS) probabilities than did the cases representing the lower 50th percentile of Hydro CSs (FIG. 13A).

The process was then repeated with three additional EBV epitopes identified to be high-confidence epitopes (Table 9) for survival distinctions: IEDB87359, IEDB134679, and IEDB86900. Similar to IEDB144799, results representing all three additional IEDB epitopes showed that cases in the upper 50th percentile of Hydro CSs had greater OS probabilities than did the cases representing the lower 50th percentile of Hydro CSs (FIGS. 13B-13D). In summary, four different B-cell EBV epitopes from IEDB indicated that high Hydro CSs were associated with greater overall survival probabilities in the NCICCR-DLBCL set.

TABLE 9
Adaptive Match web tool survival results for NCICCR-DLBCL
IGH CDR3s and 100 EBV epitopes vs. 100 non-EBV epitopes

| | | | Cox | KM |
|---|---|---|---|---|
| | Epitope Number | CS Type | p-value | p-value |
| EBV Epitopes | IEDB*144799 | Hydro | 0.0008 | 0.0195 |
| | IEDB*87359 | | 0.0064 | 0.0431 |
| | IEDB*134679 | | 0.0301 | 0.0227 |
| | IEDB*86900 | | 0.0042 | 0.0266 |
| Non-EBV | 42 | Hydro | 0.0355 | 0.0539 |
| Epitopes | 65 | | 0.0678 | 0.0918 |
| | 81 | | 0.0257 | 0.0998 |
| | 16 | | 0.0257 | 0.0998 |

Note:
Numbers for non-EBV control epitopes have been randomly assigned

### Assessment of Gender Stratification of NCICCR-DLBCL Cases Representing the Upper or Lower 50th Percentile Case Groups for Hydro CSs Based on IGH CDR3s and Multiple IEDB EBV Epitopes.

With all four of the EBV epitopes that indicated a high overall survival (OS) probability for the upper 50th percentile Hydro CS group for the NCICCR-DLBCL dataset (IEDB144799, IEDB87359, IEDB134679, and IEDB86900), there was a higher percentage of female cases in the upper 50th percentile groups of Hydro CSs in comparison to female cases in the lower 50th percentile groups, which showed a trend toward statistical significance (p-value=0.0743) (Table 10). In contrast, there was a lower percentage of male cases in the upper 50th percentile groups of Hydro CSs in comparison to male cases in the lower 50th percentile groups of Hydro CSs, which showed statistical significance (p=0.0241) (Table 10).

TABLE 10
Gender stratification between upper or lower 50th percentile
Hydro CSs case groups for the NCICCR-DLBCL IGH dataset.

| | IEDB Epitope | | | | Average |
|---|---|---|---|---|---|
| Case Groups | IEDB*144799 | IEDB*87359 | IEDB*134679 | IEDB*86900 | percent |
| Male cases in | 30 (56%) | 31 (57%) | 31 (57%) | 30 (56%) | 56.5% |
| upper 50% | | | | | |
| (Percent of | | | | | |
| cases in upper | | | | | |
| group) | | | | | |
| Male cases in | 37 (67%) | 36 (65%) | 36 (65%) | 37 (67%) | 66% |
| lower 50% | | | | | |
| (Percent of | | | | | |
| cases in lower | | | | | |
| group) | | | | | |
| Female cases in | 24 (44%) | 23 (43%) | 23 (43%) | 24 (44%) | 43.5% |
| upper 50% | | | | | |
| (Percent of | | | | | |
| cases in upper | | | | | |
| group that are | | | | | |
| female) | | | | | |
| Female cases in | 18 (33%) | 19 (35%) | 19 (35%) | 18 (33%) | 34% |
| lower 50% | | | | | |
| (Percent of | | | | | |
| cases in lower | | | | | |
| group) | | | | | |

Note:
To compare the average percentage of female cases in the upper versus lower 50th percentiles, represented by the four epitopes of indicated below, a two-proportion test was used (p = 0.0743). To compare percentage of male cases in the upper versus lower 50th percentiles, as represented by the epitopes of indicated below, a two-proportion test was used (p = 0.0241).

### Tumor Stage/Grade Representing the Upper Versus Lower 50th Percentile Hydro CSs for the TCGA-OV, NCICCR-DLBCL, and CPTAC-OV Datasets.

Chi-squared tests were completed to compare the tumor stage/grade information for the upper versus lower 50th percentile Hydro CS case groups. Tumor staging data were not available for the TCGA-OV dataset, so the TCGA-OV analysis was done with tumor-grade data. The CSs based on the EBV epitope IEDB30951 were identified as representing a statistically significant survival distinction based on the upper and lower 50th percentile of the maximal Hydro CSs (FIGS. 10A-10D). Thus, these upper and lower 50th percentile groups were used in a Chi-squared test of the potential association of the tumor grade with the IEDB30951-based Hydro CSs scores.

The Hydro CSs scores based on IEDB*30951 did not show a significant difference in tumor grade for the upper versus lower 50th percentile case groups (Chi-squared value: 1.728; 0.05 significance level critical value: 3.841; failure to reject the null hypothesis). This equates to a p-value of 0.189 (Table 11). In the above analysis of the NCICCR-DLBCL dataset, the EBV epitope IEDB144799 was shown to have statistically significant survival distinctions (FIG. 13A; Table 9). Based on Hydro CSs from IEDB144799 and NCICCR-DLBCL IGH CDR3s, there was a trending difference in tumor stage between the upper versus lower 50th percentile case groups (Chi-squared value: 3.126; 0.05 significance level critical value: 3.841; failure to reject the null hypothesis), with the upper 50th percentile group representing lower staging. This equates to a p-value of 0.077 (Table 11). In other words, NCICCR-DLBCL cases in the upper 50th percentile groups had a larger proportion of cases in the lower stages (I/II) when compared to higher stages (III/IV), which aligns with the rest of this report that demonstrates that better complementarity to EBV shows better survival outcomes.

Results of the CPTAC-OV IGH CDR3 dataset showed that tumor staging of the upper 50th percentile case group, as based on the Hydro CSs, did not show a significant difference from the lower 50th percentile Hydro CS group (Chi-squared value: 1.224; 0.05 significance level critical value: 3.841; failure to reject the null hypothesis). This equates to a p-value of 0.269. Note that no survival data were available for the CPTAC-OV dataset, so the analysis of the relationship between tumor stage and Hydro CS groups could not be based on CSs representing an EBV epitope with significant survival differences for this set. However, since the epitope IEDB*30951 showed a survival distinction in the TCGA-OV dataset with both RNAseq mining algorithms, it was also used to analyze the tumor stage between the upper and lower 50th percentile groups in the CPTAC-OV set.

In sum, Hydro CSs between the NCICCR-DLBCL IGH CDR3 dataset and a high-survival EBV epitope revealed a trending difference in tumor stage between the upper versus lower 50th percentile case groups, while the two ovarian datasets failed to show such a distinction.

TABLE 11
Chi-squared test results of tumor stage/grade based on upper
versus lower 50th percentile Hydro CSs case groups

| IEDB EBV epitope showing | Dataset | |
|---|---|---|
| a survival distinction | TCGA-OV | NCICCR-DLBCL |
| with Hydro scores | IGH | IGH |
| IEDB*30951 | Not significant | N/A |
| | (p-value = 0.189) | |
| IEDB*144799 | N/A | Trending (p-value = |
| | | 0.077) |

Note:
Trending signifies significance level <0.1. Both the IGH datasets represent the recovery of IGH recombination reads from the indicated RNAseq file datasets using the recombination read mining algorithm.
N/A, not applicable

### Discussion

This study assessed the chemical complementarity between EBV epitopes and IGH CDR3 sequences in ovarian cancer and lymphoma patients. Results indicated that higher chemical complementarity scores (CSs) were associated with better survival outcomes for both cancer types. These immunoglobulin findings are consistent with previous studies whereby patients with anti-EBV T-cell receptor CDR3s show improved survival rates for ovarian cancer and diffuse large B-cell lymphoma (DLBCL).

Considering the above results and the fact that the development of different types of lymphoma is associated with an EBV infection, these findings raise the question of whether anti-EBV IGH in the ovarian cancer setting could represent misdiagnosis of primary ovarian non-Hodgkin's lymphoma (PONHL) as ovarian epithelial carcinoma. This potential misdiagnosis might account for the improved survival rates observed in patients with higher chemical complementarity scores because lymphomas typically have a better prognosis compared to ovarian epithelial carcinoma.

The possibility of misdiagnoses in these datasets was further explored with the analysis of tumor stage/grade stratifications between upper and lower complementarity score case groups. Chi-squared tests revealed a trending difference in tumor stage between upper and lower CS groups in the NCICCR-DLBCL dataset, though no such distinction was observed in ovarian cancer datasets.

In the case of the NCICCR-DLBCL dataset, the tumor stage analysis aligned with data in the rest of this report, which showed higher survival outcomes for better IGH CDR3-EBV epitope chemical complementarity. This correlation is most likely because EBV has a clear role in the development of DLBCL. However, the ovarian dataset stage/grade analysis did not align with the IGH CDR3-EBV epitope chemical complementarity results in the rest of this report. This may be because anti-EBV IGH analysis is representative of PONHL, whereas the staging is most likely exclusively representative of ovarian epithelial carcinoma. It is important to note that there may be alternative explanations for this data, particularly in the case of the CPTAC-OV dataset, as the sample size was small and staging information was limited, with all cases used for analysis being either stage III or IV.

Additionally, gender stratification in the NCICCR-DLBCL dataset showed a higher percentage of female patients in the upper CS group, with male patients less prevalent. This indicates that females may show a better immune response and have better survival outcomes. These potential gender-related differences in survival have yet to be explored fully.

Future research could include the application of a more extensive assessment of IGH-epitope interactions. For example, a similar study utilizing molecular dynamics approaches could be envisioned. This study also highlights the potential utility of testing EBV antibody titers in the clinical setting, which need to be confirmed with prospective clinical trials. The detection of EBV immunoglobulins may support a reconsideration of PONHL in the ovarian cancer setting. In particular, a correct diagnosis is essential to make treatment decisions, which would differ significantly for patients with lymphoma versus those with ovarian epithelial carcinoma. EBV antibody titers may reflect prognoses for ovarian cancer and lymphoma patients.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred examples of the invention and that such changes and modifications can be made without departing from the spirit of the invention. It is, therefore, intended that the appended claims cover all such equivalent variations as fall within the true spirit and scope of the invention.

SEQUENCES
(TIL CDR3)
SEQ ID NO: 1
CAGGSGGGADGLTF
(TIL CDR3)
SEQ ID NO: 2
CAVSGYSTLTF
(TIL CDR3)
SEQ ID NO: 3
CAVPYNQGGKLIF
(anti-EBV CDR3)
SEQ ID NO: 4
CSARDGTGNGYTF
(anti-EBV CDR3)
SEQ ID NO: 5
CASSVGGTDTQYF
(anti-EBV CDR3)
SEQ ID NO: 6
CASSLTRTDTQYF

What is claimed is:

1. A method of determining overall survival in a subject with Epstein-Barr virus (EBV)-positive cancer, comprising:
a) obtaining a biological sample from the subject, wherein the biological sample comprises tumor-infiltrating lymphocytes (TIL);
b) extracting complementarity determining region 3 (CDR3) amino acid (AA) sequences from the TIL, thereby obtaining extracted TIL CDR3 AA sequences;
c) identifying an exact match of extracted TIL CDR3 AA sequences to known anti-EBV CDR3 AA sequences from the biological sample; thereby obtaining an exact match anti-EBV TIL CDR3 AA sequence; and
d) correlating presence of the exact match anti-EBV TIL CDR3 AA sequence with overall survival of the subject,
wherein the presence of the exact match anti-EBV TIL CDR3 AA sequence in the biological sample is correlated to higher overall survival compared to a reference control, wherein the reference control does not have the exact match anti-EBV TIL CDR3 AA sequence in the biological sample.

2. The method of claim 1, wherein the biological sample comprises blood or tumor biopsy.

3. The method of claim 1, wherein the TILs comprise B cells or T cells.

4. The method of claim 3, wherein the T cell comprises T cell receptor (TCR), wherein the TCR comprises an alpha chain or a beta chain.

5. The method of claim 3, wherein the B cell comprises B cell receptor (BCR), wherein the BCR comprises two heavy chains (IGH) and two light chains (IGL).

6. The method of claim 1, wherein the EBV-positive cancer comprises nasopharyngeal carcinoma (NPC), Hodgkin lymphoma, Burkitt lymphoma, post-transplant lymphoproliferative disorder (PTLD), gastric carcinoma, or ovarian cancer.

7. A method of determining overall survival in a subject with an EBV-positive cancer, comprising:
a) obtaining a biological sample from the subject, wherein the biological sample comprises tumor-infiltrating lymphocytes (TIL);
b) extracting complementarity determining region 3 (CDR3) amino acid (AA) sequences from the TIL, thereby obtaining extracted TIL CDR3 AA sequences;
c) obtaining a chemical complementarity score (CS) by interacting known EBV epitopes AA sequences to the extracted TIL CDR3 AA sequences;
d) calculating the CS; and
e) correlating the CS with overall survival of the subject,
wherein a high CS is correlated to better overall survival compared to a reference control, wherein the reference control has low CS.

8. The method of claim 7, wherein the CS is calculated using hydrophobic interactions, electrostatic interactions, or a combination thereof.

9. The method of claim 7, wherein the biological sample comprises blood or tumor biopsy.

10. The method of claim 7, wherein the TILs comprise B cells or T cells.

11. The method of claim 10, wherein the T cell comprises T cell receptor (TCR), wherein the TCR comprises an alpha chain or a beta chain.

12. The method of claim 10, wherein the B cell comprises B cell receptor (BCR), wherein the BCR comprises two heavy chains (IGH) and two light chains (IGL).

13. The method of claim 7, wherein the EBV-positive cancer comprises nasopharyngeal carcinoma (NPC), Hodgkin lymphoma, Burkitt lymphoma, post-transplant lymphoproliferative disorder (PTLD), gastric carcinoma, or ovarian cancer.

14. A method of treating an EBV-positive cancer in a subject, comprising:
a) obtaining a biological sample from the subject, wherein the biological sample comprises tumor-infiltrating lymphocytes (TIL);
b) extracting complementarity determining region 3 (CDR3) amino acid (AA) sequences from the TIL, thereby obtaining extracted TIL CDR3 AA sequences;
c) identifying an exact match of extracted TIL CDR3 AA sequences to known anti-EBV CDR3 AA sequences from the biological sample; thereby obtaining an exact match anti-EBV TIL CDR3 AA sequence;
d) correlating presence of the exact match anti-EBV TIL CDR3 AA sequence with overall survival of the subject; and
e) administering a pharmaceutically effective amount of an anti-EBV therapeutic to the subject with a presence of the exact match anti-EBV TIL CDR3 AA sequence,
wherein the presence of the exact match anti-EBV TIL CDR3 AA sequence in the sample is correlated to better overall survival.

15. The method of claim 14, wherein the biological sample comprises blood or tumor biopsy.

16. The method of claim 14, wherein the TILs comprise B cells or T cells.

17. The method of claim 16, wherein the T cell comprises T cell receptor (TCR), wherein the TCR comprises an alpha chain or a beta chain.

18. The method of claim 16, wherein the B cell comprises B cell receptor (BCR), wherein the BCR comprises two heavy chains (IGH) and two light chains (IGL).

19. The method of claim 14, wherein the anti-EBV therapeutic comprises an anti-viral therapeutic against EBV epitopes, an adoptive T cell therapy targeting EBV epitopes, a single-chain variable fragment (scFv) targeting EBV epitopes, or an anti-EBV antibody.

20. The method of claim 14, wherein the EBV-positive cancer comprises nasopharyngeal carcinoma (NPC), Hodgkin lymphoma, Burkitt lymphoma, post-transplant lymphoproliferative disorder (PTLD), gastric carcinoma, or ovarian cancer.
